(19) United States
(12) Patent Application Publication
(10) Pub. No.: US 2011/0054026 A1
(43) Pub. Date: Mar. 3, 2011

(54) ANTIMICROBIAL COMPOSITION AND USE

(76) Inventors: Michael Patrick Doyle, Peachtree City, GA (US); Tong Zhao, Peachtree City, GA (US)

(21) Appl. No.: 12/811,560

(22) PCT Filed: May 21, 2009
(86) PCT No.: PCT/US09/44815

§ 371 Date: Jul. 2, 2010

Related U.S. Application Data
(60) Provisional application No. 61/055,299, filed on May 22, 2008.
(60) Provisional application No. 61/085,050, filed on Jul. 31, 2008.
(60) Provisional application No. 61/151,377, filed on Feb. 10, 2009.

(51) Int. Cl. A01N 37/42; C12N 1/36; A01P 1/00

(52) U.S. Cl. 514/558; 514/557; 435/245

(57) ABSTRACT

Antimicrobial compositions are provided comprising a pharmaceutically acceptable organic acid and a pharmaceutically acceptable surfactant. This synergistic combination allows compositions to be formulated at low concentrations that have efficacy in reducing bacterial counts by greater than 3 log within 5 minutes of contact while preserving the organoleptic properties of treated foods, including fresh produce. As shown in FIG. 1C the efficacy of six different compositions (A. 3% levulinic acid plus 2% SDS; B. 2% levulinic acid plus 1% SDS; C. 0.5% levulinic acid plus 0.05% SDS; D. 3% levulinic acid; E. 2% SDS and F. water) were tested for their ability to kill spores of Bacillus anthracis Sterne after 45 minutes of contact.

### CROSS REFERENCE TO RELATED APPLICATIONS

This application claims priority under 35 USC §119(e) to U.S. Provisional Application Ser. Nos. 61/055,299 filed May 22, 2008, 61/085,050 filed Jul. 31, 2008, and 61/151,377, filed Feb. 10, 2009, the disclosures of which are incorporated herein by reference.

### BACKGROUND

Escherichia coli O157:H7 and Salmonella are major causes of severe foodborne disease in the United States and continue to be of public health significance. Salmonella is one of the most frequent causes of foodborne illnesses worldwide. In the United States, it causes an estimated 1.4 million cases of illness, approximately 20,000 hospitalizations, and more than 500 deaths annually (Mead, et al., 1999). FoodNet surveillance data of foodborne illnesses revealed that the overall incidence of salmonellosis has decreased by only 8% from 1996-1998 to 2004 and the incidence of Salmonella Enteritidis infections has remained at approximately the same level. Eating chicken is a major factor contributing to sporadic cases of Salmonella Enteritidis infections in the United States (Kimura, et al. 2004).

Other pathogens such as, for instance, Klebesiela, Proteus hauseri, Shigella, Yersinia pestis and B. anthracis, and protozoan, together with the more prominent E. coli and Salmonella, comprise a wide-spectrum of food- and water-borne pathogens which threatens the safety of the food supply and are now considered a matter of homeland security relevance. Therefore, the development of a unique, pluripotent, widely applicable, and easy to manufacture countermeasure is highly desirable.

There is growing interest in the development of novel antimicrobial treatments such as combinations of natural antimicrobials, including generally recognized as safe (GRAS) chemicals and other food preservation systems, to improve the microbiological safety of poultry products. As disclosed herein pharmaceutically acceptable chemical compositions have been formulated and have been demonstrated as having efficacy in killing large cell numbers of Salmonella on chicken skin and in chicken processing water and both Salmonella and E. coli O157:H7 on fresh produce without producing any detectable impact on the organoleptic properties of the treated food. Said composition have also been shown to be highly efficient to a large spectrum of food borne pathogens, leading to reduction of pathogen populations by factors often greater than 7 log. Time needed for reaching such level of pathogen elimination range from a few seconds to about 2 minutes. In some embodiments, reduction of pathogen population reached levels below detection limits after about 1 minute from application of compositions of the invention.

Compositions of the invention have been shown to be highly efficient in the treatment of pathogen biofilms formed on surfaces of type normally encountered on food manufacturing and processing facilities.

### SUMMARY

Applicants have discovered that several combinations of surfactants with a plurality of acids produce a synergistic effect in relation to the antimicrobial effectiveness of the individual compounds. Accordingly, this surprising synergy allows the formulation of compositions wherein the active agents (comprising an acid and a surfactant) are present at concentrations effective to reduce bacterial counts on the surface of food substances by a factor between 10³ and 10⁷ without altering the organoleptic properties of the treated food substance. In one embodiment the active agents include acids and surfactants that are FDA-approved food additives, and the treated food substances are selected from poultry, eggs, fish, seafood, meat or fresh produce.

In accordance with one embodiment a novel composition is provided comprising a pharmaceutically acceptable acid and a pharmaceutically acceptable surfactant, wherein the maximum concentration of total acid present in the composition is about 0.3 to about 3% by weight per volume in water (3-30 grams/L) and the maximum concentration of total surfactant is about 0.5% to about 1% by weight per volume in water (5-10 grams/L). In one embodiment the pharmaceutically acceptable acid is an acid that has been classified by the US Department of Agriculture as being Generally Regarded As Safe (GRAS) and includes, but is not limited to, levulinic acid, caprylic acid, caproic acid, citric acid, eugenol, adipic acid, tartaric acid, fumaric acid, lactic acid, phosphoric acid, hydrochloric acid, succinic acid, malic acid and sorbic acid.

The pharmaceutically acceptable surfactant can be selected from any ionic (cationic or anionic) or non-ionic surfactants that are compatible for human use. In accordance with one embodiment the surfactant is a functionalized organic acid having a hydrocarbon chain length of 2 to 20 carbons, wherein the functionalizing group is selected from hydroxyl, amino, carbonyl, sulphonyl, phosphate and thiol groups. Such surfactants are known to those skilled in the art in the field of food industry and include, for example, sodium dodecyl sulfate (SDS), sodium laureth sulfate (SLS; or sodium lauryl ether sulfate, SLES), cetyl pyridinium chloride (CPC), cocamide MEA (MEA), cocamide DEA (DEA), benzalkonium chloride and ethylenediamine tetraacetic acid (H₄EDTA) and its salts such as Na₄EDTA and Na₂H₂EDTA. The surfactants used may also include, in one embodiment, side group substituents attached to the hydrocarbon backbone. Such substituents can be selected from H₂PO₃, C₁-C₈ hydroxylalkyl and C₅-C₆ aryl hydroxyl. In one embodiment the surfactant is selected from the group consisting of mono-, di-, tri- and tetra-alkylammonium halides, sulfates and phosphates wherein at least one of the alkyl substituents of the alkylammonium halide comprises at least 10 carbon atoms and more typically 10-25 carbon atoms.

In one embodiment the acid selected for use in the present invention has the general structure of Formula I:

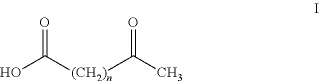

wherein n is an integer selected from 1 to 10 or 1 to 6. In one embodiment the acid comprises the structure of formula I wherein n is n is an integer selected from 1 to 3, and in another embodiment n is 1, 2 or 3. In one embodiment the acid is levulinic acid. Levulinic acid has been found to have superior qualities relative to other organic acids with regards to it ability, when used in conjunction with low concentrations of a surfactant (e.g., 0.05-2.0% w/v), to reduce viable microbe concentrations on a food by greater than 2 log within 5 minutes of exposure. Furthermore, the antimicrobial activity of the present compositions is accomplished without producing any detectable impact (by unaided human senses) on the organoleptic properties of the treated food.

In accordance with one embodiment the compositions disclosed herein may comprise two or more different acids or two or more surfactants provided that the total concentration of acid present in the composition is about 0.3% to about 3% by weight per volume in water (3-30 grams/L) and the total concentration of surfactant is about 0.5% to about 2% by weight per volume in water (5-20 grams/L). In one embodiment the total concentration of surfactant in the composition is about 0.5% to about 1% by weight per volume in water (5-10 grams/L). In accordance with one embodiment an antimicrobial composition is provided comprising levulinic acid and a surfactant, wherein the total concentration of acid in said composition is about 0.5% to about 2.0% (w/v) and the total concentration of surfactant in said composition is about 0.05% to 1% (w/v). In one embodiment the surfactant is selected from the group consisting of sodium dodecyl sulfate (SDS), sodium laureth sulfate (SLS; or sodium lauryl ether sulfate, SLES), cetyl pyridinium chloride (CPC), cocamide MEA (MEA), cocamide DEA (DEA), benzalkonium chloride and ethylenediamine tetraacetic acid (H₄EDTA). In one embodiment the surfactant is selected from the group consisting of SDS, benzalkonium chloride, and cetylpyridinium chloride.

In one specific embodiment the surfactant is SDS.

In one embodiment an antimicrobial composition is provided comprising levulinic acid and a surfactant, wherein the concentration of the levulinic acid is about 0.5% to less than 2.5% (w/v) and the concentration of the surfactant is about 0.05% to 1% (w/v). This combination, including for example levulinic acid and SDS, has been found to be particularly efficacious as an antimicrobial composition that simultaneously preserves the organoleptic properties of a treated food substance. This specific combination has been shown to be several orders of magnitude superior and/or faster in its ability o kill pathogens, than other acid/surfactant combinations. In one embodiment the surfactant is SDS. In another embodiment, an antimicrobial composition is provided comprising levulinic acid and a cationic quaternary ammonium compound, wherein the concentration of the levulinic acid is about 0.5% to about 3% and the concentration of the cationic quaternary ammonium compound is about 0.05% to 1%. In one embodiment the cationic quaternary ammonium compound is selected from the group consisting of benzalkonium chloride, cetylpyridinium bromide and cetylpyridinium chloride. The antimicrobial compositions disclosed herein are formulated at an acid pH, including for example a pH ranging from 2.5 to 3.5, and more typically a pH of 3.0 to 3.2.

The combination of a pharmaceutically acceptable acid and a surfactant have been found to exhibit a synergistic high antimicrobial activity, thus allowing for the use of low concentrations of the active agents to obtain rapid killing of large numbers of microbes upon contact. Accordingly, the low concentration compositions disclosed herein have surprising activity in reducing microbial populations on the surfaces of food items (by several log factors upon contact) without impacting the organoleptic properties of the food item. In accordance with one embodiment a method of treating a food substance to reduce resident populations of microbial and/or bacterial populations is provided. The method comprises the steps of contacting the surfaces of the food substance with a composition comprising a pharmaceutically acceptable acid and a pharmaceutically acceptable surfactant, wherein the maximum concentration of total acid present in the composition is about 0.3 to about 3% by weight per volume in water (3-30 grams/L) and the maximum concentration of total surfactant is about 0.01% to about 1% by weight per volume in water (0.1-10 grams/L). In accordance with one embodiment, the antimicrobial compositions disclosed herein are formed as a foam and the surface to be treated is contacted with the foamed composition. In accordance with one embodiment the method is used to reduce resident populations of foodborne micro-organisms including but not limited to E. coli, Salmonella, Listeria, C. botulinium, C. perfringens, C. jejuni, Giardia lamblia, C. parvum, Staphylococcus aureus, Aspergillus flavus, B. anthracis, B. cereus and Y. pestis.

In another embodiment a method is provided for the decontamination and treatment of seeds. The method comprises the step of contacting the seeds with a composition comprising levulinic acid and a surfactant. In one specific embodiment, the levulinic acid compositions of the present invention are used to treat seeds (including prophylactic treatments) to eliminate acidovorax, including for example the treatment of cucurbitaceas (e.g., watermelon) as well as in some grains (e.g. soy). In a further embodiment a method of inhibiting the growth of microbes during seed germination is provided. In this method seeds are contacted prior to, and during the germination of the seeds with a composition comprising levulinic acid and a surfactant. Surprisingly, a composition comprising about 0.3 to about 3% (w/v) levulinic acid, and 0.01 to about 1% of a surfactant has been found to be an effective antimicrobial composition that does not substantially impact seed viability or germination rates.

The presently disclosed acid/surfactant compositions can also be used to treat and inactivate bacteria present in a biofilm. In one embodiment the method comprises contacting the biofilm with an antimicrobial composition of the present disclosure, optionally in a foamed form. In one embodiment the antimicrobial composition comprises levulinic acid and a surfactant.

### BRIEF DESCRIPTION OF THE DRAWINGS

FIG. 1A-1E represent bar graphs demonstrating the efficacy of levulinic acid and SDS, alone or in combination, to kill spores of Bacillus anthracis Sterne. Spores were exposed to one of six different solutions:

A. 3% levulinic acid plus 2% SDS,

B. 2% levulinic acid plus 1% SDS,

C. 0.5% levulinic acid plus 0.05% SDS,

D. 3% levulinic acid,

E. 2% SDS,

F. water (serving as the control)

for various lengths of time (0 min., FIG. 1A; 10 min., FIG. 1B, 45 min., FIG. 1C; 90 min., FIG. 1D; 180 min., FIG. 1E), before testing the spores for viability relative to the control sample. Average plate counts are based on counting three plates; error bars indicate +/−one standard deviation.

FIG. 2A-2E represent bar graphs demonstrating the efficacy of levulinic acid and SDS, alone or in combination, to kill spores of Bacillus anthracis Sterne. Spores were exposed to one of six different solutions as disclosed in FIG. 1 for time intervals of (0 min, FIG. 2A; 1 hour, FIG. 2B, 2 hours, FIG. 2C; or 3 hours, FIG. 2D; or 4 hours, FIG. 2E), before testing the spores for viability relative to the control sample. In order to differentiate whether CFU originated from vegetative cells or from spores, at each time point samples were split in two equivalent aliquots. One aliquot was subjected to heat treatment (65° C., 30 min) to kill vegetative cells before enumeration of residual heat-resistant spores. The other aliquot was plated at room temperature (RT). Average plate counts are based on counting three plates; error bars indicate +/−one standard deviation.

FIG. 3A-3E represent bar graphs demonstrating the efficacy of levulinic acid and SDS, alone or in combination, to kill spores of Bacillus anthracis Sterne. Spores were exposed to one of six different solutions as disclosed in FIG. 1 for time intervals of (0 min., FIG. 3A; 1 hour, FIG. 3B, 2 hours, FIG. 3C; or 3 hours, FIG. 3D; or 4 hours, FIG. 3E), before testing the spores for viability relative to the control sample. In order to differentiate whether CFU originated from vegetative cells or from spores, at each time point samples were split in two equivalent aliquots. One aliquot was subjected to heat treatment (65° C., 30 min) to kill vegetative cells before enumeration of residual heat-resistant spores. The other aliquot was plated at room temperature (RT). Average plate counts are based on counting three plates; error bars indicate +/−one standard deviation.

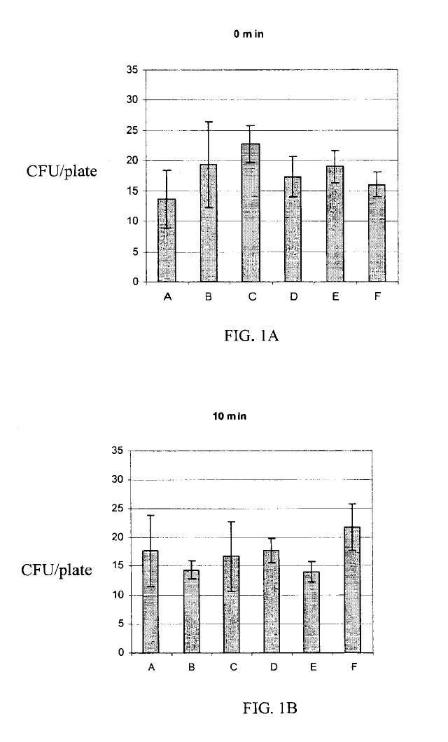

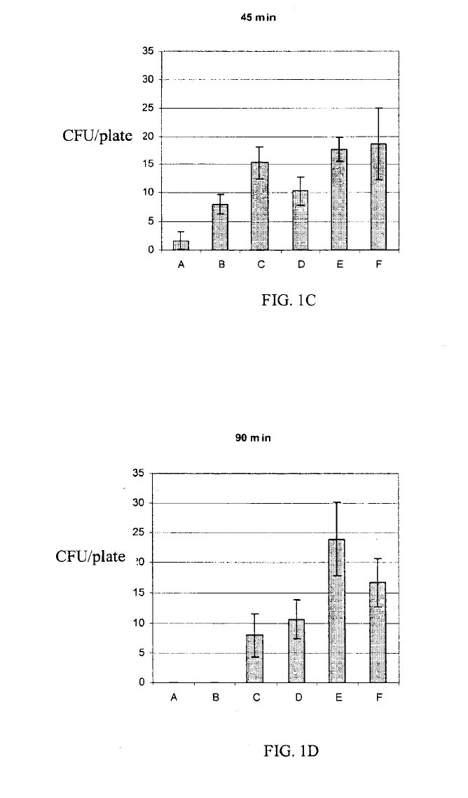

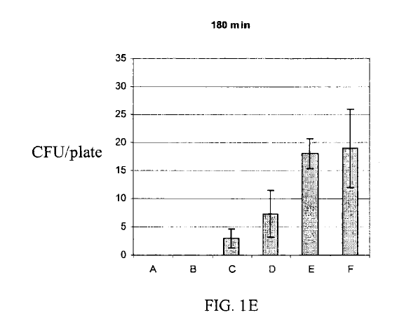

### DETAILED DESCRIPTION

### Definitions

In describing and claiming the invention, the following terminology will be used in accordance with the definitions set forth below.

As used herein the term “microorganism” or “microbe” is intended to include living cellular organisms, both unicellular and multicellular that are less than 5 mm in length, and include but are not limited to bacteria, fungi, archaea, protists; green algae, plankton, planarian, amoebas and yeasts, or spores formed by any of these.

As used herein an “antimicrobial” is a compound that exhibits microbicidal or microbiostatic properties that enables the compound to kill, destroy, inactivate, or neutralize a microorganism; or to prevent or reduce the growth, ability to survive, or propagation of a microorganism.

As used herein the term “acid” refers to any chemical compound that, when dissolved in water, gives a solution with a hydrogen ion activity greater than in pure water, i.e. a pH less than 7.0. An “organic acid” is a carbon containing compound (except for carbonic acid) with acidic properties.

A monoprotic acid is an acid that is able to donate one proton per molecule during ionization.

The term “about” as used herein means greater or lesser than the value or range of values stated by 1/10 of the stated values, but is not intended to limit any value or range of values to only this broader definition. For instance, a concentration value of about 30% means a concentration between 27% and 33%. Each value or range of values preceded by the term “about” is also intended to encompass the embodiment of the stated absolute value or range of values.

As used herein, the term “pharmaceutically acceptable” is intended to encompass any compound that can be safely administered to warm blooded vertebrates including humans. Pharmaceutically acceptable acids and surfactants include acids and surfactants that are classified by the United States Food and Drug Administration (FDA) as being Generally Regarded As Safe (GRAS), and encompass any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

A quaternary ammonium cation is a compound of the general structure:

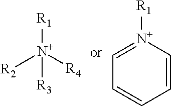

wherein R₁, R₂, R₃, and R₄ are independently selected from the group consisting of C₁-C₂₀ alkyl and salts thereof.

As used herein the term “benzalkonium chloride” refers to a single alkylbenzyldimethylammonium chloride of the general structure

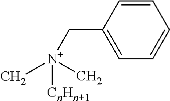

wherein n is an integer selected from the group consisting of 6, 8, 10, 12, 14, 16, 18 and 20, or mixtures of two or more such compounds.

As used herein an “effective” amount or a “therapeutically effective amount” of an anti-microbial composition refers to a concentration of active agent that provides the desired effect, i.e., log order reduction in surface microbial counts on a food substance without reducing organoleptic properties of the food substance.

As used herein the term “germination” refers to the initiation of growth of an embryonic plant contained within a seed, through completion of establishment of the seedling, wherein the seedling has exhausted the food reserves stored in the seed.

As used herein “organoleptic properties” relating to properties that can be detected by human or animal senses (taste, color, odor, feel) unaided by mechanical and analytical devices.

As used herein a “food substance” relates to any material that is edible by mammals, including for example, a human.

As used herein reference to a “cylinder foam test” is intended to refer a test for measuring both the foamability of compositions and the persistence of the foamed state. In general, the test comprises the steps of placing a test composition into a stoppered, graduated cylinder so that the composition occupies a predetermined height of the cylinder (e.g., about ⅓ to about ½ of the height of the stoppered, graduated cylinder). The stoppered, graduated cylinder is then inverted approximately 10 times to generate a foam. The height of foam is measured immediately after the inverting step as a measure of the foamability of the composition. The foamed composition is then left undisturbed to determine the foam half life (time required for the foam to lose half its height in the graduated cylinder). The cylinder foam test is conducted at room temperature under 1 standard atmosphere pressure (i.e., 101.3 kPa (about 760.01 mmHg) or 29.92 in Hg).

### EMBODIMENTS

An antimicrobial composition is provided herein comprising a pharmaceutically acceptable acid and a pharmaceutically acceptable surfactant. Surprisingly, the compositions disclosed herein are capable of reducing resident microbial populations on the surface of food substance by a factor greater than 10², including by a factor of 10³ to a factor of 10⁸, using a combination of an acid and surfactant at concentrations that are ineffective when used separately. The individual active ingredients of the present compositions (i.e., the pharmaceutically acceptable acid and surfactant) are ineffective in reducing microbial cell count by a factor greater than 10², even when the active agents are used separately at 2× or 5× the effective concentration used in the combination. In one embodiment the concentration of the pharmaceutically acceptable acid in the antimicrobial composition is within the range of about 0.03% to about 3%, or about 0.05% to about 2%, or about 0.05% to about 1%, or about 0.1% to about 3%, or about 0.3% to about 3%, or about 0.3% to about 2%, or about 0.5% to about 3%, or about 0.5% to about 2%, or about 0.5% to about 1%, weight per volume in water. In one embodiment the concentration of the pharmaceutically acceptable surfactant in the antimicrobial composition is within the range of about 0.005% to about 1%, or about 0.01% to about 1%, or about 0.05% to about 1%, or about 0.1% to about 1%, or about 0.05% to about 2%, or about 0.5% to about 2% by weight per volume in water.

In accordance with one embodiment an antimicrobial composition is provided comprising a linear monoprotic organic acid and an ionic long chain (C₈-C₃₀) surfactant. In one embodiment the organic acid is a linear monoprotic organic acid comprising a carbon backbone of 4 to 10 or 4 to 6 carbons. In one embodiment an antimicrobial composition is provided comprising a pharmaceutically acceptable acid and a surfactant, wherein of the general structure of the acid is CH₃(CH₂)ₘCOOH, with m being an integer selected from 2-8, and the surfactant is selected from the group consisting of sodium dodecyl sulfate (SDS), sodium laureth sulfate (SLS; or sodium lauryl ether sulfate, SLES), cetyl pyridinium chloride (CPC) and benzalkonium chloride. In one embodiment the composition comprises an acid of the general structure CH₃(CH₂)ₘCOOH, with m being an integer selected from 2-8 or 4-8 and the surfactant is selected from the group consisting of sodium dodecyl sulfate (SDS) and sodium laureth sulfate (SLS; or sodium lauryl ether sulfate, SLES). In another embodiment the composition comprises an acid of the general structure CH₃(CH₂)ₘCOOH, or

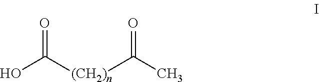

wherein m is an integer selected from 2-8 or 4-8 and n is an integer selected from 1 to 10 or 1 to 6, and the surfactant is a cation of the general structure:

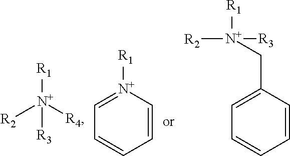

wherein R₁, R₂, R₃, and R₄ are independently selected from the group consisting of C₁-C₂₀ alkyl, and salts thereof. In one embodiment R₁ is C₆-C₂₀ alkyl and R₂, R₃, and R₄ are independently selected from the group consisting of C₁-C₂ alkyl.

In accordance with one embodiment the organic acid is selected from the group consisting of eugenol, hexanoic acid, levulinic acid, succinic acid. In one embodiment the acid component of the antimicrobial composition consists of an acid having the general structure of Formula I:

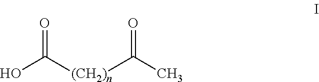

wherein n is an integer selected from 1 to 10 or 1 to 6. In one embodiment the acid comprises the structure of formula I wherein n is n is an integer selected from 1 to 3, and in another embodiment n is 1, 2 or 3. In one embodiment the surfactant is selected from the group consisting of benzalkonium halide, cetypridinium chloride, cetypridinium bromine, and SDS. In one embodiment the composition comprises one of the following combinations:

1) 0.05% to 2.0% (w/v) eugenol plus 0.05% to 1.0% (w/v) SDS;

2) 0.05% to 2.0% (w/v) hexanoic acid plus 0.05% to 1.0% (w/v) SDS

3) 0.05% to 2.0% (w/v) levulinic acid plus 0.05% to 1.0% (w/v) benzalkonium chloride;

4) 0.05% to 2.0% (w/v) levulinic acid plus 0.05% to 1.0% (w/v) cetypridinium chloride;

5) 0.05% to 1.0% (w/v) succinic acid plus 0.05% to 1.0% (w/v) SDS. In one embodiment the composition comprises 0.5% eugenol plus 0.05% SDS (pH 3.2), 0.5% hexanoic acid plus 0.05% SDS (pH 3.2), 0.5% levulinic acid plus 0.05% benzalkonium chloride (pH 3.1), 0.5% levulinic acid plus 0.05% cetypridinium chloride (pH 3.1) or 0.5% succinic acid plus 0.05% SDS (pH 2.9), or combinations thereof.

Previous studies revealed that combinations of different organic acids can be used as anti-bacterial agents based on their killing effects on E. coli O157:H7 and Campylobacter (Zhao, et al. 2006). Levulinic acid is an organic acid that can be produced cost effectively and in high yield from renewable feedstocks (Bozell, et al. 2000, Fang and Hanna, 2002). Its safety for humans has been widely tested and FDA has given it GRAS status for direct addition to food as a flavoring agent or adjunct (21 CFR, 172.515). Its application to fresh produce may extend shelf life because levulinic acid can arrest light-induced chloroplast development during greening and can be removed by washing the leaves to restore the developmental process (Jilani, et al. Physiol. Plantarum (1996) 96:139-145).

As disclosed herein, the bactericidal effect of 1% by weight levulinic acid alone will not suffice to kill more than 1 log CFU Salmonella/ml within 30 minutes, and its bactericidal effect was increased only to 3.4 log CFU/ml within 30 minutes when the levulinic acid concentration was increased to 3% by weight (see Tables 1-3). Sodium dodecyl sulfate (SDS) also has GRAS status (21 CFR, 172.210) at 0.5% wt of gelatin, as a whipping agent in gelatin used in marshmallows and at 0.0125% in liquid and frozen egg whites. It has been widely studied and is used as a surfactant in household products such as toothpastes, shampoos, shaving foams, and bubble baths. The SDS molecule has a tail of 12 carbon atoms attached to a sulfate group, giving the molecule the amphiphilic properties required of a surfactant. As disclosed herein the use of SDS by itself has very little bactericidal effect (see Tables 1-3).

As reported herein, combining a pharmaceutically acceptable surfactant with a pharmaceutically acceptable acid synergistically enhances the antimicrobial activity of the respective surfactant and acid. In accordance with one embodiment the pharmaceutically acceptable acid is selected from the group consisting of levulinic acid, caprylic acid, caproic acid, citric acid, eugenol, adipic acid, tartaric acid, fumaric acid, lactic acid, phosphoric acid, succinic acid, malic acid and sorbic acid. The pharmaceutically acceptable surfactant in one embodiment is selected from any ionic (cationic or anionic) or non-ionic surfactants that are compatible for human use. Such surfactants are known to those skilled in the art in the field of food industry and include, for example, sodium dodecyl sulfate (SDS), sodium laureth sulfate, cetyl pyridinium chloride (CPC), cocamide MEA (MEA), cocamide DEA (DEA), benzalkonium chloride and ethylenediamine tetraacetic acid (EDTA). In accordance with one embodiment the surfactant is an anionic surfactant, such as SDS, and the acid is an organic acid selected from the group consisting of caprylic acid, levulinic acid, lactic acid and acetic acid. SDS when combined with organic acids dramatically increased the bactericidal effect of organic acid treatments. The substantial bactericidal effect of a combination of levulinic acid and SDS on E. coli O157:H7 and Salmonella was validated on fresh produce, poultry wings, chicken skin and water containing different levels of chicken feces or feathers (see Example 1, Tables 4-7). In addition, the bactericidal activity of this combination of chemicals remained effective even in an organic-rich environmental containing fecal matter or feathers.

In accordance with one embodiment an antimicrobial composition is provided. The composition comprises pharmaceutically acceptable surfactant and a pharmaceutically acceptable organic acid, wherein the concentration of the organic acid is 0.5% by weight/volume or less and the concentration of the surfactant is 0.05% by weight/volume or less. In one embodiment the pharmaceutically acceptable surfactant is an anionic surfactant. As used herein the term organic acid refers to a compound having a hydrocarbon chain and an acid group covalently bound to the hydrocarbon chain. The hydrocarbon chain can be of any length and can be a straight chain or branched chain. The most common organic acids are the carboxylic acids whose acidity is associated with their carboxyl group —COOH. However, additional compounds that lack a carboxylic function group can still function as an acid in accordance with the present invention if the compound ionizes in aqueous solution to yield hydrogen ions. Accordingly, eugenol is considered an acid within the context of the present invention due to the electron withdrawing properties of the phenol ring on the hydroxyl group substitutent. Sulfonic acids, containing the group OSO₃H, are another typical, but relatively stronger group of organic acids. In accordance with one embodiment the organic acid is a carboxylic acid comprising a maximum of 2 to 10 carbon atoms. The organic acids used in the present invention may also include additional functional groups extending from the hydrocarbon backbone. In one embodiment the carbon chain of the organic acid is functionalized by a hydroxyl, a carbonyl, an amino, an alkylamino, a sulfonyl, or a thiol group.

The surfactant used in the compositions of the present disclosure may be selected from any of the known organic surfactants (i.e., organic compounds that are amphiphilic, containing both hydrophobic groups and hydrophilic groups), including, ionic (cationic or anionic) and non-ionic surfactants, or mixtures thereof. In one embodiment the surfactant is an ionic surfactant, and more typically an anionic surfactant. In one embodiment the surfactant is an anionic surfactant comprising a 10 to 20 length carbon chain linked to the hydrophilic head group. In one embodiment the surfactant is an organic phosphate or sulfate wherein the carbon chain of said organic phosphate or sulfate comprises 12 carbon atoms. In one embodiment the surfactant is SDS. In accordance with one embodiment the composition comprises a maximum concentration of 0.3 to 3% by weight of one or more organic acids selected from the group consisting of lactic acid, acetic acid, and levulinic acid and a maximum concentration of 0.05 to 2% by weight SDS. In one embodiment the composition comprises 0.3 to 3% by weight levulinic acid and 0.05 to 1% by weight SDS.

The antimicrobial compositions disclosed herein can be used to reduce the population of an undesirable microbe on an object, including food substances. For the purpose of this patent application, successful reduction of a population of a microbe is achieved when the populations of the microbe is reduced by at least 2 log. In accordance with one embodiment, the method comprises contacting the object with a composition comprising levulinic acid and a pharmaceutically acceptable surfactant. In one embodiment the composition is a foam composition. The foamed composition can be formed as part of the administration/contacting step, using any of a variety of foaming apparatus known to those skilled in the art, such as a portable foamer or an aspirating wall mounted foamer.

In accordance with one embodiment the antimicrobial compositions are used to treat a food processing surface. As used herein, the phrase “food processing surface” refers to a surface of a tool, a machine, equipment, a structure, a building, or the like that is employed as part of a food processing, preparation, or storage activity. Examples of food processing surfaces include surfaces of food processing or preparation equipment (e.g., slicing, canning, or transport equipment, including flumes), of food processing wares (e.g., utensils, dishware, wash ware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners and sanitizers, ware washing, blancher cleaning, food packaging materials, cutting boards, beverage chillers and warmers, meat chilling or scalding equipment, cooling towers, food processing garment areas (including drains). Advantageously, the present compositions have been found to remain effective even in an organic-rich environmental containing fecal matter or feathers. Thus the compositions can be used as a single wash treatment of surfaces that may contain such materials in addition to pathogenic microbes.

In accordance with one embodiment an antimicrobial composition comprising levulinic acid and a surfactant is provided wherein the composition is effective in reducing resident microbial populations on food substance. In one embodiment, a food contaminated with 10⁸-10⁹ CFU/ml E. coli O157:H7 can be treated with the antimicrobial compositions disclosed herein to reduce the presence of viable bacteria by a factor greater than 10³ (including reductions of 10⁴, 10⁵, 10⁶ and 10⁷ or even higher) after exposure to said composition for five minutes, under conditions otherwise favorable to proliferation of said E. coli O157:H7. In one embodiment the concentration of said levulinic acid and surfactant are at concentrations that are ineffective in reducing said resident microbial population when used separately. In one embodiment the concentration of each of the levulinic acid and surfactant components is at a concentration 0.5×, 0.25×, 0.1×, or less than 0.1×, of the concentration required to produce a significant reduction (e.g., greater than one log reduction within 5 minutes) in an E. coli O157:H7 microbial population when the respective component (i.e., levulinic acid or surfactant) is used separately. In one embodiment the concentration of the levulinic acid in the compositions of the present invention is no more than 3%, 2.5%, 2.0%, 1.5%, 1.0%, 0.5% or 0.25% (w/v). In one embodiment the concentration of the levulinic acid is less than 2.5% (w/v) or less than 2.0% (w/v) and in a further embodiment the concentration of the levulinic acids is about 0.5% (w/v) levulinic acid. These concentrations of levulinic acid in combination with a pharmaceutically acceptable surfactant at concentrations of less than 2% have been found to retain the organoleptic properties of foods, including produce. The concentration of the surfactant in one embodiment of the present compositions is no more than about 0.01% to about 1%, or about 0.01% to about 0.1% and more typically is about 0.05% (w/v).

In one embodiment the surfactant is a sulfate, sulfonate or carboxylate anion and in another embodiment the surfactant is a quaternary ammonium cation. In one embodiment the quaternary ammonium cation is benzalkonium chloride, cetylpyridinium bromide or cetylpyridinium chloride.

In another embodiment a method for the rapid killing of microbial strains is provided. The method comprises contacting bacteria with a composition comprising a surfactant and an organic acid, wherein the concentration of the organic acid is 3.0%, 2.0%, 1.0% or 0.5% (w/v) or less and the concentration of the surfactant is less than 1%, 0.5%, 0.1% or 0.05% (w/v). In one embodiment the organic acid is selected from the group consisting of lactic acid, acetic acid, and levulinic acid and the surfactant is an anionic surfactant, including for example SDS. In accordance with one embodiment the composition comprises levulinic acid and SDS, and in a further embodiment the composition comprises a maximum concentration of 0.3 to 3% by weight levulinic acid and a maximum concentration of 0.05 to 1% by weight SDS. In one embodiment, the organic acid/SDS compositions disclosed herein are used to inactivate bacterial strains including pathogenic strains of Salmonella and E. coli. The treatments can be conducted at temperatures favorable to retaining the desirable properties of fresh produce, including at temperatures of 20-25° C. or 20-22° C.

In accordance with one embodiment the surface to be treated is contacted with the levulinic acid containing solution by any standard technique, including spraying, washing, immersion, rinsing, soaking (with or without agitation) and similar methods known to those skilled in the art. Advantageously, applicants have found that by spraying the present compositions under relatively low pressure, the composition will be applied as a foam. For example using a composition comprising 2% SDS and a simple weed sprayer, the composition is applied as a foam that is comparable to that when a foaming agent is needed for applying disinfectants to equipment and environmental surfaces in food processing facilities. The foam persists for at least 20 minutes if left undisturbed. In one embodiment the pressure used to produce consistant form (e.g., one that lasts for 20 minutes) for a 3% levulinic acid plus 2% SDS (w/v) is 15 to 35 psi. The concentration of the active agents can be reduced to 2% levulinic acid and 1% SDS and formation of a consistent foam can still be obtained using a similar pressure. The use of a foamed form of the composition is advantageous as it allows for better penetration of the active agents on the treated surface.

When the present composition is provided as a foam, the composition has a cellular structure that can be characterized as having several layers of air cells that provide the composition with a foamy appearance. It should be understood that the characterization of a foam refers to the existence of more than simply a few air bubbles and in one embodiment the foam retains over 20, 30, 40, 50, 60 or 70% of its maximum height in a cylinder foam test 10 minutes after agitation ceases. In one embodiment the foamed antimicrobial composition of the present disclosure retains at least 20% of its height in a cylinder foam test 5 minutes after agitation is ceased.

The cylinder foam test has been used in the surfactant industry to evaluate the foamability of test compositions. In general, the cylinder foam test is conducted by adding a test composition to a stoppered, graduated cylinder so that the composition occupies a predetermined height of the cylinder (e.g., about ⅓ to about ½ of the height of the stoppered, graduated cylinder). The stoppered, graduated cylinder is inverted approximately 10 times and the height of foam generated can be recorded. The persistence of the foam can be determined by measuring the height of the foamed composition in the graduated cylinder over time in the absence of further agitation. The test is typically conducted under room temperature under standard atmospheric conditions.

Typically, the antimicrobial compositions disclosed herein can be formed as a foam using simple mechanical foaming heads known to those skilled in the art that function by mixing air and the composition to create a foamed composition. However, the use of known chemical foaming mechanisms is also suitable for forming foams in accordance with the present invention. For chemical foaming, the antimicrobial composition can include ingredients that create foam as a result of a chemical interaction, either with other ingredients in the composition, or with substances present in the applicable environment. These components can be provided as a 2-part composition that can be combined when foaming is desired.

Foaming can be accomplished, for example, using a foam application device such as a tank foamer or an aspirated wall mounted foamer, e.g., employing a foamer nozzle of a trigger sprayer. For example, foaming can be accomplished by placing the composition in a fifteen gallon foam application pressure vessel, such as a fifteen gallon capacity stainless steel pressure vessel with mix propeller. The foaming composition can then be dispensed through a foaming trigger sprayer. A wall mounted foamer can use air to expel foam from a tank or line.

The antimicrobial compositions disclosed herein can be optionally administered to a food substance or a food processing surface as a foam. The foam can be prepared by mixing air with the antimicrobial composition through use of a foam application device. Mechanical foaming heads that can be used according to the invention to provide foam generation include those heads that cause air and the foaming composition to mix and create a foamed composition. That is, the mechanical foaming head causes air and the foaming composition to mix in a mixing chamber and then pass through an opening to create a foam.

Suitable mechanical foaming heads that can be used according to the invention include those available from Airspray International, Inc. of Pompano Beach, Fla., and from Zeller Plastik, a division of Crown Cork and Seal Co. Suitable mechanical foaming heads that can be used according to the invention are described in, for example, U.S. Pat. No. D-452,822; U.S. Pat. No. D-452,653; U.S. Pat. No. D-456,260; and U.S. Pat. No. 6,053,364. Mechanical foaming heads that can be used according to the invention includes those heads that are actuated or intended to be actuated by application of finger pressure to a trigger that causes the foaming composition and air to mix and create a foam. That is, a person's finger pressure can cause the trigger to depress thereby drawing the foaming composition and air into the head and causing the foaming composition and air to mix and create a foam.

In accordance with one embodiment additional foam boosting agents are added to the antimicrobial compositions to enhance either foamability and/or longevity of the formed foam. In accordance with one embodiment the antimicrobial compositions disclosed herein further comprise a foam boosting solvents selected from the group consisting of glycols, glycol ethers, derivatives of glycol ethers, and mixtures thereof. Suitable glycols include those having at least four carbon atoms such as hexylene glycol.

In one embodiment, a food substance or an object in a food processing environment can be treated with the antimicrobial compositions. In accordance with one embodiment a method is provided for preparing a processed food with antibacterial qualities. The food is combined with an antimicrobial composition disclosed herein using any standard technique, including for example, spraying, immersion, rinsing, soaking, injecting, washing and the like. Optionally, the food can be more rigorously mixed with the antimicrobial compositions by use of stirring, grinding, pulverizing, macerating, or other known techniques, to produce the combined food and antimicrobial composition. In accordance with one embodiment the antimicrobial composition comprises an organic acid having the general structure of:

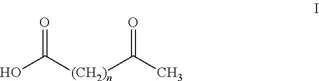

wherein n is an integer selected from 1 to 6, and a surfactant selected from the group consisting of a quaternary ammonium cation, sodium dodecyl sulfate, sodium laureth sulfate, and cetyl pyridinium chloride. Such an antimicrobial composition is combined with a food raw material component to form a mixture. The mixture is then optionally subjected to further processing to form said processed food. In one embodiment the antimicrobial composition component comprises levulinic acid and SDS. In a further embodiment the method comprises combining the antimicrobial composition with unprocessed meats and then grinding the combined components. In another embodiment the food comprises shelved nuts, wherein after combination of the nuts with the antimicrobial composition, the combined components are then ground for the preparation of nut butters. Other foods including fish and seafood can similarly be combined with the presently disclosed antimicrobial compositions. In a further embodiment the antimicrobial compositions disclosed herein can be used as an additive to solutions packaged with a food.

In accordance with one embodiment an antimicrobial composition is provided comprising an organic acid and an anionic surfactant, wherein the maximum concentration of the acid in the composition is about 0.3 to about 3% by weight per volume in water (3-30 grams/L) and the maximum concentration of total surfactant is about 0.01% to about 1% by weight per volume in water (0.1-10 grams/L). In one embodiment the organic acid is levulinic acid, and the surfactant is sodium dodecyl sulfate (SDS). In accordance with one embodiment an antimicrobial composition is provided comprising levulinic acid and a cationic surfactant, wherein the maximum concentration of the acid in the composition is about 0.3 to about 3% by weight per volume in water (3-30 grams/L) and the maximum concentration of total surfactant is about 0.01% to about 1% by weight per volume in water (0.1-10 grams/L). In one embodiment the compositions comprise further antimicrobial agents known to those skilled in the art. For example the compositions may further comprise one or more antimicrobial agents selected from the group consisting of antibiotics, hydrogen peroxide and alcohols.

As disclosed herein a group of organic acids, including lactic acid, acetic acid, and levulinic acid, were evaluated individually or in combination with sodium dodecyl sulfate (SDS) to kill Salmonella. Results revealed that these chemicals, if used individually at 0.5% by weight for the organic acid or 0.05% by weight for SDS, inactivated ≦2 log CFU/ml within 20 minutes at 21° C. Combining any of these organic acids at 0.5% by weight with 0.05% by weight SDS resulted in the surprising result of >7 log CFU/ml inactivation of Salmonella within 10 seconds. Accordingly, as disclosed herein harmful microorganisms (such as Salmonella and E. coli O157::H7 at 10⁸CFU/ml) can be killed rapidly by treatment with levulinic acid plus SDS. Combinations of different concentrations of levulinic acid (0.3 to 3% by weight in water) plus SDS (0.05 to 1% by weight in water) were evaluated for killing E. coli O157:H7 and Salmonella on lettuce and spinach. Results revealed that E. coli O157:H7 or Salmonella populations on either lettuce or spinach or tomato were reduced by greater than 4 log CFU/g after receiving this treatment for 5 minutes at 21° C.

Additional tests were done on chicken skin contaminated with Salmonella and in water containing chicken feathers or feces. Results revealed that Salmonella cell numbers on chicken skin were reduced by more than 5 log CFU/cm² after treatment with as little as 0.5% by weight levulinic acid plus 0.05% by weight SDS for 5 minutes, on poultry wings with 3% by weight levulinic acid plus 2% by weight SDS, and in water containing chicken feathers or feces with 1% by weight levulinic acid plus 0.1% by weight SDS. The use of levulinic acid in combination with SDS as a wash solution is highly desirable because of its surprising efficacy in killing foodborne pathogens, low cost, and environmentally friendly nature.

Processing equipment is commercially available for washing produce (and processing other foods), and applicants have found that the levulinic compositions of the present invention (eg. compositions having a concentration up to 3% levulinic acid) is not corrosive to such equipment. In particular, applicants have found that using a large stainless steel seed washing unit provided by a seed supplier, not only was the levulinic acid treatment as effective in killing E. coli O157:H7 as the gold standard 20,000 ppm calcium hypochlorite, but it was not corrosive to the equipment and even removed rust on chains within the unit. Thus the levulinic acid composition served to clean the unit like a detergent without the undesirable corrosive effect on equipment that is associated with many sanitizers such as chlorine. Accordingly, one embodiment of the present invention is also directed to a method of decontaminating equipment and hard surfaces by contacting such equipment and hard surfaces with the levulinic compositions of the present invention. In accordance with one embodiment a foaming composition is provided comprising 0.5% to 3.0% (w/v) levulinic acid and 1.0 to 3.0% (w/v) SDS. In one embodiment the foaming composition comprises 0.5% to 3.0% (w/v) levulinic acid and 2.0% (w/v) SDS. Furthermore, a composition comprising 3% levulinic acid plus 1% SDS can come in contact with skin without the irritation caused by other organic acids.

In a further embodiment a method for rapid killing of microbial strains present in liquids or on surfaces contaminated with feces and/or other animal fluids (e.g., urine or saliva) or animal materials (e.g. feathers, hair) is also provided. The method comprises contacting the liquid or surface with a composition comprising an organic acid, selected from the group consisting of lactic acid, acetic acid, and levulinic acid, and SDS, wherein the composition comprises a maximum concentration of 3% by weight levulinic acid and 2% by weight SDS. In one embodiment the composition used comprises levulinic acid and a surfactant.

The reduction of pathogens, including Salmonella and E. coli O157:H7, resulting from the use of the compositions disclosed herein is a log reduction (>5 log/ml or greater within one minute), not a percent reduction as reported and approved by prior art formulations of organic acids. The bactericidal effects of organic acids have been documented. However these prior art formulations have never been USDA approved for application. The main reasons include doubtable bactericidal results when applied in the product lines, sensory or surface color changes of the treated products, short shelf-life, cost control and difficulty with regards to management or practice. The mere percentage reduction obtained with the prior art formulations, such as for instance, those obtained through the use of citric acid, is simply too little, and thus such compositions fail to provide an efficient or reliable means for safeguarding foods. The present compositions represent the first reliable approach to eliminate Salmonella from the poultry products and E. coli O157:H7 from the meat and fresh produce.

In accordance with one embodiment a method of reducing resident microbial populations on the surface of a food is provided. In one embodiment the food to be treated is selected form the group consisting of produce, meat, eggs, seafood and fish. The method comprises the step of contacting a food or a food processing surface with a composition comprising levulinic acid and a surfactant, wherein the concentration of each of said levulinic acid and surfactant is at a concentration 0.5×, 0.25×, 0.1×, or less than 0.1× of the concentration required to produce a significant reduction (e.g., greater than one log reduction within 5 minutes) in an E. coli O157:H7 microbial population when used separately.

In one embodiment the surface of the food is contacted with the levulinic acid/surfactant containing solution for a predetermined length of time, including lengths of time of 1, 2, 3, 4, 5 or 10 minutes. Applicants have established that such exposure times can be used without negatively impacting the organoleptic properties of the food. Such time interval have been found to be effective in reducing viable cell counts by at least 3 orders of magnitude. More particularly, applicants have demonstrated that compositions comprising levulinic acid, at a concentration of 3% (w/v) or less, in combination with a surfactant (such as SDS) reduces viable microbe cells counts by a factor of 5 to ≧7 logs within 1 to 5 minutes of contact under conditions otherwise suitable for microbe growth.

In one embodiment the antimicrobial formulations disclosed herein comprise a combination of levulinic acid at a concentration of 0.5% to 3% weight/volume plus a surfactant such as a quaternary ammonium cation or SDS at a concentration of 0.05% to 2% weight/volume. In one embodiment a foamed antimicrobial formulations is provided comprising levulinic acid, at a concentration of 0.5% to 3% weight/volume plus a pharmaceutically acceptable surfactant at a concentration of 0.05% to 3% by weight/volume. Additional combinations of levulinic acid and a surfactant (e.g., SDS) at different concentrations relative to one another will be prepared based on the desired application. For example, three specific combinations will be developed for treatment of different products. Lower concentration (0.5% by weight levulinic acid plus 0.05% by weight SDS) will be selected for treatment of fragile products, such as spinach, lettuce, tomato and sprouts. Middle concentration (2% by weight levulinic acid plus 1% by weight SDS) will be selected for treatment of vegetables and fruits. Relatively higher concentration (3% by weight levulinic acid plus 2 to 3% by weight SDS) will be selected for treatment of meats, food processing surfaces, and environmental samples, such cages, traffic areas, and transportation vehicles. Fish and seafood can be treated with any of the three concentrations of levulinic acid and SDS as mentioned immediately above. In one embodiment the fish or seafood is treated with a middle concentration (2% by weight levulinic acid plus 1% by weight SDS) of the antimicrobial composition. The compositions will also be formulated as different washing solutions, such as washing for all meats, washing for seafood, washing for fish, washing for vegetables, washing for fruits, and washing for environmental samples.

Formulations based on levulinic acid are cheap, easy to produce, do not produce bad odor, release to environment is friendly, plus studies have been performed in human health area (it is widely added in cigarettes for reduction of nicotine). Both levulinic acid and SDS have been approved for use in food by FDA.

In accordance with one embodiment the antimicrobial compositions of the present invention can be used to remove biofilms from a solid surface, including for example, a food processing surface. The method comprises contacting the biofilm with the antimicrobial composition, optionally in the form of a foamed composition. In one embodiment the biofilm is contacted with an aqueous composition comprising 0.5% to 3% by weight per volume in water of an organic acid and 0.05% to 2% by weight per volume in water of an ionic surfactant. In one embodiment the organic acid is a monoprotic organic acid comprising a carbon backbone of 4 to 10 or 4 to 6 carbons. More particularly, in one embodiment the organic acid has the general structure of:

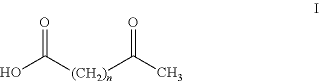

wherein n is an integer selected from 1 to 10 or 1 to 6. In one embodiment the acid comprises the structure of formula I wherein n is n is an integer selected from 1 to 3, and in another embodiment n is 1, 2 or 3. In one embodiment the surfactant is selected from the group consisting of benzalkonium halide, cetypridinium chloride, cetypridinium bromine, and SDS. In accordance with one embodiment the antimicrobial composition comprises levulinic acid and sodium dodecyl sulfate and/or sodium laureth sulfate. In one embodiment the concentration of the levulinic acid is less than 3%, 2.5%, 2.0%, 1.5%, 1.0%, 0.5% or 0.25% (w/v) of the aqueous composition and the concentration of the sodium dodecyl sulfate and/or sodium laureth sulfate is less than 2.0, 1.5, 1.0, 0.5, 0.1 or 0.05% (w/v) of the aqueous composition.

The present antimicrobial compositions can also be use in accordance with one embodiment in a method of treating seeds to remove pathogenic microbes from seeds. The method comprises contacting the seeds with the antimicrobial composition, optionally in the form of a foamed composition. In one embodiment the biofilm is contacted with an aqueous composition comprising 0.5% to 3% by weight per volume in water of an organic acid and 0.05% to 2% by weight per volume in water of an ionic surfactant. In one embodiment the organic acid is a monoprotic organic acid comprising a carbon backbone of 4 to 10 or 4 to 6 carbons. More particularly, in one embodiment the organic acid has the general structure of:

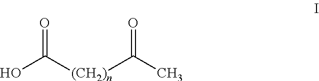

wherein n is an integer selected from 1 to 10 or 1 to 6. In one embodiment the acid comprises the structure of formula I wherein n is n is an integer selected from 1 to 3, and in another embodiment n is 1, 2 or 3. In one embodiment the surfactant is selected from the group consisting of benzalkonium halide, cetypridinium chloride, cetypridinium bromide, and SDS. In accordance with one embodiment the antimicrobial composition comprises levulinic acid and sodium dodecyl sulfate and/or sodium laureth sulfate.

In accordance with one embodiment a method of decontaminating seeds is provided comprising the steps of contacting the seeds with a composition comprising levulinic acid and a surfactant, wherein the concentration of each of said levulinic acid and surfactant present in said composition is at a concentration 0.5×, 0.25×, 0.1×, or less than 0.1×, of the concentration required to produce a significant reduction (e.g., greater than 50% reduction) in an E. coli O157:H7 microbial population when used separately. In one embodiment the concentration of the levulinic acid is less than 3%, 2.5%, 2.0%, 1.5%, 1.0%, 0.5% or 0.25% (w/v) of the aqueous composition. In one embodiment the concentration of the levulinic acid is less than 2.5% (w/v) and in a further embodiment the concentration of the levulinic acids is about 0.5% levulinic acid. Furthermore, the concentration of the surfactant is no more than about 0.01% to about 2%, or about 0.01% to about 0.1% and more typically is about 0.05% (w/v). This treatment can be used to eliminate pathogenic organisms such as E. coli O157:H7, Salmonella, Bacillus anthracis, B cereus and Acidovorax avenae from seeds, and have shown efficacy for killing the spores of such organisms.

In one embodiment of the invention, a solution comprising levulinic acid and a surfactants such as SDS, can be added to food items such ground meats, pastes and butters, during the process of manufacturing of said food items, thus providing for an intimate mixture between the food items and the antimicrobial of the invention, thus enhancing the safety and shelf-life of those products.

The levulinic compositions have also been added to water used during seed germination and results indicate it does not adversely affect germination. Thus in addition to treating the seeds, the present levulinic acid compositions could be used to eliminate any residual pathogenic organisms (such as E. coli O157:H7 and Salmonella) that survive an initial treatment of seeds with either 20,000 ppm calcium hypochlorite or the levulinic acid compositions of the present invention. This will further safeguard against the possibility of pathogens surviving initial seed treatments and prevent grow of pathogenic organisms in the germination medium. In accordance with one embodiment a method of inhibiting the growth of microbes during seed germination is provided wherein the method comprises contacting the seeds prior to, and during the germination of the seeds with a composition comprising levulinic acid and a surfactant. In one embodiment the composition comprises less than 3% levulinic acid and less than 1% of a surfactant.

### Example 1

### Microbiocidal Efficacy of the Organic Acid/SDS Compositions

### Materials and Methods

Strains. Five isolates of E. coli O157:H7, including 932 (human isolate), E009 (beef isolate), E0018 (cattle isolate), E0122 (cattle isolate), E0139 (deer jerky isolate); and five isolates of Salmonella Typhmurium DT104, including three cattle isolates and two meat isolates; and five isolates of Salmonella Enteritidis, including 564-88 (food isolate), 193-88 (human isolate), E39 (egg isolate), 460-88 (egg isolate) and 457-88 (poultry isolate); and five isolates of L. monocytogenes, including LM101 (serotype 4b, salami isolate), LM 112 (serotype 4b, salami isolate), LM113 (serotype 4b, pepperoni isolate), LM9666 (serotype 1/2c, human isolate), and LM5779 (serotype 1/2 c, cheese isolate); and one isolate of Yersinia pestis (A1122) were used. Each Salmonella and E. coli O157:H7 strain was grown in tryptic soy broth (TSB) at 37° C. for 18 h then washed in 0.1 M phosphate buffered saline pH 7.2. Approximately equal cell numbers of each of the five strains were combined and used as a 5-strain mixture with cell numbers being adjusted according to the experimental design. Bacterial cell numbers were confirmed by serial dilutions (1:10) in 0.1% peptone and a volume of 0.1 ml from each dilution tube was plated on tryptic soy agar (TSA), XLD agar, and Sorbitol MacConkey agar (SMA), incubated at 37° C. for 24 h, and colonies were counted.

Chemicals and chemical treatment. Acetic acid, caprylic acid, lactic acid, levulinic acid and sodium dodecyl sulfate (SDS) were tested alone or as a combination at different concentrations and temperatures (8 or 21° C.) for their killing effect on S. enteritidis, S. Typhimurium, and E. coli O157:H7 in water contaminated with chicken feces or feathers with and without feces and on chicken skin with and without chicken feces.

Fresh produce. Romaine lettuce, tomato and spinach were purchased from a local retail store. Prior to each study, the produce was tested for Salmonella. A volume of 10 ml of sterile water and 10 g lettuce or spinach was added to a Whirl-Pak bag. The sample bag was pummeled in a stomacher blender at 150 rpm for 1 min. The fluid was serially (1:10) diluted in 0.1% peptone and 0.1 ml from each dilution tube was plated in duplicate on XLD plates to determine if these samples were contaminated with Salmonella. Only Salmonella-negative lettuce, tomato and spinach were used.

Chicken feathers, skin, poultry wings and feces. Feces from a poultry farm was collected from 5 different chickens and used as a mixture. Feathers were obtained from a slaughterhouse. Chicken and poultry wings were purchased from a slaughter plant or local retail store and skin was separated immediately before use. Only Salmonella-negative chicken feces, feather, skin, or poultry wing samples were used for the experiments. A volume of 10 ml of deionized water and 1.0 g feces, or feathers, or a piece of skin (5×5 cm²) was added to a Whirl-Pak bag. Each bag of feces, feather, or skin sample was pummeled in a stomacher blender at 150 rpm for 1 min. The bag of poultry wing was massaged by hands for 1 min. The fluid was serially (1:10) diluted in 0.1% peptone and 0.1 ml from each dilution tube was plated in duplicate on XLD plates to determine if these samples were contaminated with salmonellae. Only Salmonella-negative chicken feces, feather, or skin samples were selected for experiments.

Enumeration of S. enteritidis, S. Typhimurium DT104 and E. coli O157:H7. At each sampling time, 1.0 ml of the treated bacterial suspension was mixed with 9.0 ml of neutralizing buffer or PBS (depending on the pH). The solution was serially (1:10) diluted in 0.1% peptone water and 0.1 ml of each dilution was surface-plated onto TSA and XLD, or TSA and XLD containing ampicillin (32 μg/ml), tetracycline (16 μg/ml) and streptomycin (64 μg/ml) (TSA+, XLD+), or TSA and Sorbitol MacConkey agar plates in duplicate. The plates were incubated at 37° C. for 48 h. Colonies typical of Salmonella or E. coli O157:H7 were randomly picked from plates with the highest dilution for confirmation of Salmonella or E. coli by biochemical tests and for confirmation of serotyping by latex agglutination assay. When Salmonella or E. coli O157:H7 were not detected by direct plating, a selective enrichment in universal preenrichment broth (UPB) was performed by incubating 25 ml of treatment suspension in a 500 ml flask containing 225 ml of UPB for 24 h at 37° C. Following pre-enrichment, 1 ml was transferred to 10 ml of selenite cystine broth and incubated for 24 h at 37° C. Following incubation, a 10-μl loopful from the broth tube was plated in duplicate onto XLD plates, and incubated for 24 h at 37° C. Colonies with typical Salmonella spp. morphology were selected and transferred one more time on XLD plates and incubated for 24 h at 37° C. All presumptive Salmonella isolates were tested by the Salmonella latex agglutination assay. Isolates positive for Salmonella by the latex agglutination assay were tested with the API 20E assay for biochemical characteristics for the identification of Salmonella. Studies with all chemical treatments were done in duplicate or triplicate, two replicates were plated per sample and results were reported as means.

Determination of Salmonella and E. coli O157:H7 inactivation on lettuce or spinach. Samples of 25 g Romaine lettuce were cut in ca. 5-cm length in a laminar flow hood. Whole tomatoes (150 g±10 g) were used. The samples were soaked in E. coli O157:H7 or Salmonella (10⁸-10⁹ CFU/ml) suspension for 60 sec and then air-dried for 20 minutes for lettuce and spinach, 60 minutes for tomato in a laminar hood. The samples were then soaked in a 1000-ml glass beaker containing 500 ml chemical solution or 500-ml glass beaker containing 200 ml chemical solution with agitation at 100 rpm by a magnetic bar at 21° C. Following treatment, the sample was placed in a stomacher bag containing 10 ml PBS and pummeled for 1 minute at 150 rpm in a stomacher or in a shaker. The solution was serially (1:10) diluted in 0.1% peptone and a volume of 0.1 ml from each dilution tube was plated on the surface of TSA and XLD for S. Enteritidis, TSA and XLD, XLD+ for S. Typhimurium DT 104 and TSA and SMA for E. coli O157:H7 in duplicate for bacterial enumeration.

Determination of Salmonella inactivation in water contaminated with chicken feathers or feces. The protocols used were the same as described previously (Zhao, et al. 2006), with minor modifications. Chicken feathers or feces were weighed and added into a glass beaker containing chemicals to be determined according to different ratios (w/v) in a glass beaker and mixed by a magnetic bar with agitation at 150 rpm. A 5-strain mixture of S. enteritidis was added. A volume of 1 ml sample was removed and serially diluted (1:10) in PBS. The aerobic bacterial and Salmonella counts were determined according to the procedures we described above.

Determination of Salmonella inactivation on poultry wings. Chicken wings (each ca. 12 cm long, 7 cm wide, and ca. 85 to 90 g) were submerged in a glass beaker containing 500 ml of S. enteritidis (ca. 10⁸ CFU/ml) for 60 sec. Inoculated wings were air dried for 20 min in a laminar flow hood and then individually placed in a Whirl-Pak bag containing 200 ml of chemical solution for 0, 1, 2, 5, 10, 20, 30, and 60 min. The bags were agitated in a vertical shaker at 150 rpm with intermittent hand massage. Following chemical treatment, each chicken wing was placed in a Whirl-Pak bag containing 50 ml of 0.1 M PBS. The bag was agitated in a vertical shaker for 2 min at 150 rpm with intermittent hand massage. The cell suspension (1 ml) was serially (1:10) diluted in 9 ml of 0.1% peptone, and 0.1 ml portions of each dilution was surface plated in duplicate on XLD and TSA plates. The plates were incubated at 37° C. for 24 and 48 h to enumerate the bacterial number.

Determination of Salmonella inactivation on chicken skin. Chicken skin was separated and cut into a 5×5 cm² square per sample immediately before the experiment. S. enteritidis at 10⁷-10⁸ CFU with and without feces were inoculated onto the skin and air-dried under a laminar flow hood for 20 minutes. The inoculated skin was placed into a stomacher bag containing the antimicrobial solution (200 ml solution for each skin sample) at 21° C. for a contact time of 0, 1, 3, 5, 10, and 20 minutes with hand massage intermittently (every 30 seconds) or pummeled by a stomacher at 150 rpm. The samples were placed in Whirl-Pak bags, each containing 9 ml PBS then pummeled in a stomacher blender at 150 rpm for 1 min. Salmonella were enumerated according to the procedures described above.

### Results

Determination of Salmonella inactivation in water with 0.1 to 2.0% by weight levulinic acid revealed about a 1-log CFU/ml reduction. Its killing effect was greater when the levulinic acid concentration was increased to 3.0% by weight, resulting in a 3.4-log Salmonella/ml reduction when in contact for 30 minutes (Table 1). Treatments of 0.5% by weight acetic acid and 0.5% by weight lactic acid for 30 minutes reduced Salmonella cell numbers by 0.7- and 2.0-log CFU/ml, respectively. A treatment of 0.05% by weight SDS for 30 minutes did not reduce Salmonella cell numbers (Table 1).

All the combinations of organic acids evaluated in combination with 0.03-0.05% by weight SDS were effective, at different degrees, in killing Salmonella, with the population of Salmonella quickly reduced from 10⁷ CFU/ml to undetectable (enrichment-negative) with a contact time of 5-10 seconds (see Table 1).

Neither levulinic acid at 0.5% by weight nor SDS at 0.05% by weight when applied individually provided a significant killing effect on either E. coli O157:H7 or S. Typhimurium DT 104; however, the combination of levulinic acid and SDS at these concentrations reduced E. coli O157 and S. Typhimurium cell numbers by 7 log CFU/ml within 1 min (see Tables 2 & 3).

The antimicrobial activity of levulinic acid and SDS on Salmonella on fresh produce and chicken skin was determined. Results revealed that S. enteritidis cell numbers on lettuce were reduced by ca. 4 log CFU/g when treated for 1 min with 0.3% by weight levulinic acid plus 0.05% by weight SDS, and S. typhimurium on lettuce or spinach was reduced by ca. 4 log CFU/g when treated for 1 min with 0.5% by weight levulinic acid and 0.05% by weight SDS, respectively. E. coli O157:H7 on lettuce was reduced by 4.5 log CFU/g when treated for 1 min with 0.5% by weight levulinic acid and 0.05% by weight SDS (see Table 4). When the concentration of levulinic acid was increased to 3% by weight and SDS to 1% by weight, their antimicrobial activity on lettuce also increased. All inoculated E. coli O157:H7 and S. typhimurium cells were inactivated to undetectable levels within 1 min with this treatment (Table 4).

Studies with chicken skin revealed S. enteritidis was reduced by 6.3 log CFU/g when treated for 5 min with 0.5% by weight levulinic acid and 0.05% by weight SDS (Table 4).

Both Salmonella and E. coli O157:H7 were undetectable by the direct plating method in the chemical solutions after they were used for treatment of fresh produce or chicken skin (Table 4).
The levulinic acid and SDS treatment to kill S. enteritidis was further tested in water containing chicken feathers or feces. Results revealed that feather contamination did not reduce the killing effect of that treatment, whereas the presence of chicken feces did. S. enteritidis was reduced from 7.6 log CFU/ml to 1.2 log CFU/ml in chicken feces contaminated water after 2 min exposure, but was not detected (7.6 log CFU/ml reduction) after 5 min (P<0.05; Table 5). Greater concentrations of levulinic acid and SDS were more effective in killing Salmonella, even in water heavily contaminated with chicken feces (1 part feces: 20 parts water; wt/v) (Table 5).

Studies on S. enteritidis on poultry wings revealed that treatment with a solution of 3% by weight levulinic acid and 2% by weight SDS inactivated all inoculated Salmonella (>6 log CFU/ml reduction) within 1 min. At the same time the total microbial population was also reduced by this treatment for >7 log CFU/ml (Table 7).

Aerobic bacteria counts in water contaminated with chicken feces at a ratio of 1:100 (w/v) were reduced by >4.0 log CFU/ml after treatment with 1% by weight levulinic acid and 0.1% by weight SDS for 2 min. The antimicrobial effect was increased to ca. 5.5 log CFU/ml reduction in water contaminated with chicken feces at a ratio of 1:20 (w/v) when the chemical concentrations were increased to 3% by weight levulinic acid plus 2.0% by weight SDS for 2 min (Table 6).

As disclosed herein a combination of two chemicals, which includes an organic acid (classified as generally recognized as safe by FDA, including lactic acid, acetic acid, levulinic acid, caprylic acid, et al.) and sodium dodecyl sulfate (SDS, the anionic surfactant compound) can be used to kill harmful bacterial present on food substances and/or food processing surfaces. In one embodiment the chemical combination comprises 45 mM levulinic acid and 1.73 mM SDS, which can rapidly (within 8 seconds) kill up to 7 log of pathogens, including Yersinia pestis, Salmonella Enteritidis, S. Typhimurium DT104, Listeria monocytogenes, and Escherichia coli O157:H7. Levulinic acid (45 mM plus SDS (1.73 mM) reduced S. Enteritidis, S. Typhimurium DT104 and E. coli O157:H7 in fresh produce (lettuce and spinach) by 5 logs as fast as within 15 seconds. This chemical combination is stable at room temperature and environmentally friendly. There is no apparent organoleptic difference between fresh produce treated with this chemical solution for up to 60 minutes and fresh produce treated with water or without treatment. Users of this type of product are fresh produce and poultry processors and individual households to reduce Salmonella and E. coli O157:H7.

TABLE 1
Reduction of S. Enteritidis in water treated
with different organic acids and SDS at 21° C.

| | S. Enteritidis counts (log CFU/ml) at min: | | | | | |
|---|---|---|---|---|---|---|
| Chemical Treatment | 0 | 2 | 5 | 10 | 20 | 30 |
| S. Enteritidis only (pH 6.7) | 7.2 | 7.0 | 7.1 | 7.2 | 7.0 | 7.2 |
| (Control) | | | | | | |
| 0.1% levulinic acid (pH 2.5) | 7.1 | 7.1 | 6.9 | 7.0 | 6.9 | 6.9 |
| 0.5% levulinic acid (pH 2.6) | 7.1 | 6.8 | 6.9 | 6.9 | 6.6 | 6.7 |
| 1.0% levulinic acid (pH 2.9) | 6.9 | 6.7 | 6.8 | 6.9 | 6.9 | 6.7 |
| 1.5% levulinic acid (pH 2.8) | 6.7 | 6.7 | 6.8 | 6.7 | 6.4 | 6.5 |
| 2.0% levulinic acid (pH 2.8) | 6.7 | 6.7 | 6.7 | 6.8 | 6.5 | 6.0 |
| 2.5% levulinic acid (pH 2.6) | 6.9 | 6.8 | 6.9 | 6.4 | 5.8 | 4.8 |
| 3.0% levulinic acid (pH 2.7) | 6.6 | 6.8 | 6.5 | 6.2 | 5.1 | 3.8 |
| 0.5% acetic acid (pH 3.1) | 7.1 | 7.0 | 6.8 | 6.7 | 6.6 | 6.5 |
| 0.5% lactic acid (pH 2.6) | 6.5 | 6.1 | 5.9 | 5.8 | 5.5 | 5.2 |
| 0.05% sodium dodecyl | 7.1 | 7.0 | 7.2 | 7.1 | 7.2 | 7.1 |
| sulfate (pH 4.4) | | | | | | |
| 0.3% levulinic acid + | −^{a} | − | − | − | − | − |
| 0.05% SDS (pH 3.1) | | | | | | |
| 0.4% levulinic acid + | − | − | − | − | − | − |
| 0.05% SDS (pH 2.9) | | | | | | |
| 0.5% levulinic acid + | − | − | − | − | − | − |
| 0.05% SDS (pH 3.0) | | | | | | |
| 0.5% levulinic acid + | − | − | − | − | − | − |
| 0.03% SDS (pH 3.0) | | | | | | |
| 0.05% caprylic acid + | − | − | − | − | − | − |
| 0.03% SDS (pH 3.4) | | | | | | |
| 0.05% caprylic acid + | − | − | − | − | − | − |
| 0.05% SDS (pH 3.2) | | | | | | |
| 0.5% acetic acid + | − | − | − | − | − | − |
| 0.05% SDS (pH 3.0) | | | | | | |
| 0.5% lactic acid + | − | − | − | − | − | − |
| 0.05% SDS (pH 2.5) | | | | | | |

^{a}−, negative by enrichment culture.

TABLE 2
Reduction of E. coli O157:H7 in water treated
with levulinic acid and SDS at 21° C.

| | E. coli O157:H7 counts (log CFU/ml) at min: | | | | | | | |
|---|---|---|---|---|---|---|---|---|
| Chemical Treatment | 0 | 1 | 2 | 5 | 10 | 20 | 30 | 60 |
| E. coli O17:H7 only | 7.1 | 7.2 | 7.0 | 7.2 | 7.1 | 7.1 | 7.2 | 7.2 |
| (Control) | | | | | | | | |
| 0.5% levulinic acid | 7.0 | 6.7 | 6.8 | 6.7 | 6.9 | 6.8 | 6.8 | 6.4 |
| (pH 3.0) | | | | | | | | |
| 0.05% SDS (pH 7.0) | 7.1 | 6.9 | 7.1 | 7.0 | 6.9 | 6.9 | 7.1 | 7.0 |
| 0.5% levulinic acid | −^{a} | − | − | − | − | − | − | − |
| plus 0.05% SDS | | | | | | | | |
| (pH 3.0) | | | | | | | | |

^{a}−, negative by enrichment culture

TABLE 3
Reduction of S. Typhimurium DT 104 in water
treated with levulinic acid and SDS at 21° C.

| | S. Typhimurium DT 104 counts | | | | | | | |
|---|---|---|---|---|---|---|---|---|
| | (log CFU/ml) at min: | | | | | | | |
| Chemical Treatment | 0^{a} | 1 | 2 | 5 | 10 | 20 | 30 | 60 |
| S. Typhimurium only | 6.9 | 7.0 | 7.0 | 7.0 | 7.0 | 6.9 | 7.0 | 7.0 |
| (Control) | | | | | | | | |
| 0.5% levulinic acid | 6.8 | 6.7 | 6.6 | 6.5 | 6.7 | 6.6 | 6.4 | 5.9 |
| (pH 3.0) | | | | | | | | |
| 0.05% SDS (pH 7.0) | 7.0 | 7.0 | 6.8 | 6.9 | 6.8 | 6.9 | 6.9 | 6.9 |
| 0.5% levulinic acid | +^{a} | −^{b} | − | − | − | − | − | − |
| plus 0.05% SDS | | | | | | | | |
| (pH 3.0) | | | | | | | | |

^{a}+, positive by enrichment (minimum detection level is 0.7 log CFU/ml)
^{b}−, negative by enrichment culture

TABLE 4
S. Enteritidis, E. coli O157:H7 and S. Typhimurium
DT 104 counts for levulinic acid plus SDS treatment
on fresh produce or chicken skin at 21° C.

| | S. Enteritidis counts (log CFU/ml) at min: | | | | |
|---|---|---|---|---|---|
| | | | | | In treatment |
| | | | | | solution |
| Treatment | 0 | 1 | 2 | 5 | (5 min) |
| Romaine Lettuce Treatment | | | | | |
| S. Enteritidis in | 7.7 | 7.3 | 7.4 | 7.3 | 7.4 |
| lettuce only | | | | | |
| 0.3% levulinic acid + | 3.1 | 3.1 | 2.7 | 2.6 | <0.7^{a} |
| 0.05% SDS (pH 3.1) | | | | | |
| S. Typhimurium DT 104 on | 7.4 | 7.3 | 7.4 | 7.3 | 7.4 |
| lettuce treated with PBS | | | | | |
| 0.5% levulinic acid + | 2.8 | 2.9 | 2.9 | 2.7 | <0.7 |
| 0.05% SDS (pH 3.1) | | | | | |
| treatment | | | | | |
| 3% levulinic acid + | <0.7 | <0.7 | <0.7 | <0.7 | <0.7 |
| 1% SDS (pH 2.7) | | | | | |
| treatment | | | | | |
| E. coli O157:H7 in lettuce | 7.4 | 7.5 | 7.2 | 7.2 | 7.4 |
| treated with PBS | | | | | |
| 0.5% levulinic acid + | 3.1 | 3.0 | 3.0 | 2.9 | <0.7 |
| 0.05% SDS (pH 3.0) | | | | | |
| treatment | | | | | |
| 3% levulinic acid + | <0.7 | <0.7 | <0.7 | <0.7 | <0.7 |
| 1% SDS (pH 2.7) | | | | | |
| treatment | | | | | |
| Spinach Treatment | | | | | |
| S. Typhimurium DT 104 on | 8.0 | 7.9 | 8.1 | 7.9 | 7.9 |
| spinach treated with PBS | | | | | |
| 0.5% levulinic acid + | 4.3 | 3.8 | 4.4 | 4.7 | <0.7 |
| 0.05% SDS (pH 3.0) | | | | | |
| treatment | | | | | |
| Chicken skin Treatment | | | | | |
| S. Enteritidis on chicken | 7.1 | 7.3 | 7.2 | 7.0 | 6.8 |
| skin only | | | | | |
| 0.5% levulinic acid + | 6.7 | 4.4 | 3.5 | 0.7 | <0.7 |
| 0.05% SDS (pH 3.0) | | | | | |

^{a}Minimum detection level by direct plating method.

TABLE 5
S. Enteritidis counts for treatment of levulinic acid plus
SDS in water containing chicken feathers or feces at 21° C.

| | S. Enteritidis counts (log CFU/ml) at min: | | | | | |
|---|---|---|---|---|---|---|
| Treatment | 0 | 2 | 5 | 10 | 20 | 30 |
| In water containing chicken | | | | | | |
| feathers (1:100, w/v) | | | | | | |
| S. Enteritidis (pH 6.7) only | 7.5 | 7.7 | 7.4 | 7.5 | 7.6 | 7.6 |
| 1.0% levulinic acid + | <0.7^{a} | <0.7 | <0.7 | <0.7 | <0.7 | <0.7 |
| 0.1% SDS (pH 3.2) | | | | | | |
| In water containing chicken | | | | | | |
| feces (1:100, w/v) | | | | | | |
| S. Enteritidis only (pH 6.8) | 7.6 | 7.5 | 7.5 | 7.6 | 7.5 | 7.6 |
| 1.0% levulinic acid + | 4.9 | 1.2 | <0.7 | <0.7 | <0.7 | <0.7 |
| 0.1% SDS (pH 4.0) | | | | | | |
| In water containing chicken | | | | | | |
| feces (1:20, w/v) | | | | | | |
| S. Enteritidis only (pH 6.7) | 7.7 | 7.8 | 7.7 | 7.7 | 7.7 | 7.6 |
| 3.0% levulinic acid + | <0.7 | <0.7 | <0.7 | <0.7 | <0.7 | <0.7 |
| 2.0% SDS (pH 4.0) | | | | | | |

^{a}Minimum detection level by direct plating method

TABLE 6
Aerobic bacteria counts for treatment of levulinic acid
plus SDS in water containing chicken feces at 21° C.

| | Aerobic bacteria counts | | | | | |
|---|---|---|---|---|---|---|
| | (log CFU/ml) at min: | | | | | |
| Treatment | 0 | 2 | 5 | 10 | 20 | 30 |
| In water containing chicken | | | | | | |
| feces (1:100, w/v) | | | | | | |
| Aerobic bacteria only | 7.4 | ND^{a} | ND | 7.4 | 7.4 | 7.4 |
| 1.0% levulinic acid + | 5.0 | 3.0 | 2.9 | 2.9 | 2.0 | 2.0 |
| 0.1% SDS (pH 4.0) | | | | | | |
| In water containing chicken | | | | | | |
| feces (1:20, w/v) | | | | | | |
| Aerobic bacteria only | 10.4 | 10.4 | 10.3 | 10.4 | 10.4 | 10.4 |
| 3.0% levulinic acid + | 4.5 | 4.9 | 5.1 | 4.9 | 5.1 | 5.1 |
| 2.0% SDS (pH 4.0) | | | | | | |

^{a}ND, Not determined.

TABLE 7
Salmonella and aerobic bacteria counts for treatment
of levulinic acid plus SDS on poultry wings at 8° C.

| | | | | | In treatment |
|---|---|---|---|---|---|
| | | | | | solution |
| Treatment | 0 | 1 | 2 | 5 | (5 min) |
| | S. Enteritidis counts (log CFU/ml) at min: | | | | |
| PBS (7.2) treatment | 6.5 | ND^{a} | ND | 6.5 | 7.6 |
| 3% levulinic acid + | 6.1 | <0.7^{b} | <0.7 | <0.7 | <0.7 |
| 2% SDS (pH 2.7) | | | | | |
| treatment | | | | | |
| | Aerobic bacteria counts (log CFU/ml) at min: | | | | |
| PBS (pH 7.2) treatment | 7.9 | N/A | N/A | 8.5 | 9.8 |
| 3% levulinic acid + | 7.8 | <0.7 | <0.7 | <0.7 | <0.7 |
| 2% SDS (pH 2.7) | | | | | |
| treatment | | | | | |

^{a}ND, not determined
^{b}Minimum detection level by direct plating method

TABLE 8
Counts of S. Enteritidis on chicken wings treated with levulinic acid plus
SDS at 8° C.

| | Means (±SD) bacterial counts (log CFU/cm2) at minute: | | | |
|---|---|---|---|---|
| | | | | In treatment solution |
| Treatment | 0 | 1 | 5 | (5 min) |
| S. Enteritidis only | 7.8 ± 0.0 | 7.0 ± 0.2 | 6.8 ± 0.1 | 7.3 ± 0.1 |
| 2.0% levulinic acid + 1.0% | 7.3 ± 0.2 | 4.4 ± 0.1 | 3.2 ± 0.2 | + |
| SDS | | | | |
| S. Enteritidis only | 7.4 ± 0.1 | 6.7 ± 0.4 | 7.0 ± 0.2 | 6.9 ± 0.1 |
| 3.0% levulinic acid + 1.0% | 7.4 ± 0.2 | 2.7 ± 0.1 | 2.2 ± 0.2 | − |
| SDS | | | | |
| S. Enteritidis only | 6.5 ± 0.5 | 6.7 ± 0.4 | 6.5 ± 0.3 | 7.6 ± 0.0 |
| 3.0% levulinic acid + 2% SDS | 6.1 ± 0.2 | + | − | − |

+, positive by enrichment culture but not by direct plating (minimum detection level is 1.7 log CFU/ml)
−, negative by direct plating and enrichment culture

TABLE 9
Effect of a combination with 0.5% levulinic acid and 0.05%
SDS, pH 3.1 at 21° C. on different bacterial species

| | Bacterial counts (log CFU/ml) at min: | | | | | | | |
|---|---|---|---|---|---|---|---|---|
| Bacterial Name | 0^{a} | 1 | 2 | 5 | 10 | 20 | 30 | 60 |
| Klebsiella pneumonia in 0.1 M | ND^{b} | ND | ND | 6.5 | ND | ND | ND | 6.6 |
| PBS (Control) | | | | | | | | |
| Klebsiella pneumonia in 0.5% | −^{c} | − | − | − | − | − | − | − |
| levulinic acids plus 0.05% SDS | | | | | | | | |
| (pH 3.1) | | | | | | | | |
| Hafinia alvei in 0.1 M PBS | ND | ND | ND | 6.9 | ND | ND | ND | 6.9 |
| (control) | | | | | | | | |
| Hafinia alvei in 0.5% levulinic | − | − | − | − | − | − | − | − |
| acids plus 0.05% SDS (pH 3.1) | | | | | | | | |
| Klebsiella oxytoca in 0.1 M | ND | ND | ND | 7.2 | ND | ND | ND | 7.1 |
| PBS (Control) | | | | | | | | |
| Klebsiella oxytoca in 0.5% | − | − | − | − | − | − | − | − |
| levulinic acids plus 0.05% SDS | | | | | | | | |
| (pH 3.1) | | | | | | | | |
| Proteus hauseri in 0.1 M PBS | ND | ND | ND | 7.3 | ND | ND | ND | 7.4 |
| (Control) | | | | | | | | |
| Proteus hauseri in 0.5% | − | − | − | − | − | − | − | − |
| levulinic acids plus 0.05% SDS | | | | | | | | |
| (pH 3.1) | | | | | | | | |
| Serratia marcesens in 0.1 M | ND | ND | ND | 7.3 | ND | ND | ND | 7.3 |
| PBS (Control) | | | | | | | | |
| Serratia marcesens in 0.5% | − | − | − | − | − | − | − | − |
| levulinic acids plus 0.05% SDS | | | | | | | | |
| (pH 3.1) | | | | | | | | |
| Shigella flexneri in 0.1 M PBS | ND | ND | ND | 7.1 | ND | ND | ND | 7.1 |
| (Control) | | | | | | | | |
| Shigella flexneri in 0.5% | − | − | − | − | − | − | − | − |
| levulinic acids plus 0.05% SDS | | | | | | | | |
| (pH 3.1) | | | | | | | | |
| Shigella sonnei in 0.1 M PBS | ND | ND | ND | 7.3 | ND | ND | ND | 7.3 |
| (Control) | | | | | | | | |
| Shigella sonnei in 0.5% | − | − | − | − | − | − | − | − |
| levulinic acids plus 0.05% SDS | | | | | | | | |
| (pH 3.1) | | | | | | | | |
| Staphylococcus aureus in 0.1 M | ND | ND | ND | 6.9 | ND | ND | ND | 6.9 |
| PBS (Control) | | | | | | | | |
| Staphylococcus aureus in 0.5% | − | − | − | − | − | − | − | − |
| levulinic acids plus 0.05% SDS | | | | | | | | |
| (pH 3.1) | | | | | | | | |
| Aerococcus viridans in 0.1 M | ND | ND | ND | 6.0 | ND | ND | ND | 6.0 |
| PBS (control) | | | | | | | | |
| Aerococcus viridans in 0.5% | − | − | − | − | − | − | − | − |
| levulinic acids plus 0.05% SDS | | | | | | | | |
| (pH 3.1) | | | | | | | | |
| Yersinia pseudotubersulosis in | ND | ND | ND | 7.0 | ND | ND | ND | 7.0 |
| 0.1 M PBS (control) | | | | | | | | |
| Yersinia pseudotubersulosis in | − | − | − | − | − | − | − | − |
| 0.5% levulinic acids plus | | | | | | | | |
| 0.05% SDS (pH 3.1) | | | | | | | | |
| E. coli O26:H11 in 0.1 M PBS | ND | ND | ND | 7.2 | ND | ND | ND | 7.2 |
| (Control) | | | | | | | | |
| E. coli O26:H11 in 0.5% | − | − | − | − | − | − | − | − |
| levulinic acids plus 0.05% SDS | | | | | | | | |
| (pH 3.1) | | | | | | | | |
| E. coli O111:NM in 0.1 M PBS | ND | ND | ND | 7.1 | ND | ND | ND | 7.1 |
| (Control) | | | | | | | | |
| E. coli O111:NM in 0.5% | − | − | − | − | − | − | − | − |
| levulinic acids plus 0.05% SDS | | | | | | | | |
| (pH 3.1) | | | | | | | | |
| Vibro chloerae in 0.1 M PBS | ND | 5.1 | 5.0 | ND | ND | ND | 4.2 | ND |
| (control) | | | | | | | | |
| Vibro chloerae in 0.5% | − | − | − | − | − | − | − | − |
| levulinic acids plus 0.05% SDS | | | | | | | | |
| (pH 3.1) | | | | | | | | |
| Campylobacter jejuni in 0.1 M | 8.2 | 8.3 | 8.1 | 8.0 | 8.4 | 8.1 | 8.2 | 8.4 |
| PBS (control) | | | | | | | | |
| Campylobacter jejuni in 0.5% | <0.7 | <0.7 | <0.7 | <0.7 | <0.7 | <0.7 | <0.7 | <0.7 |
| levulinic acids plus 0.05% SDS | | | | | | | | |
| (pH 3.1) | | | | | | | | |

(ND = Not Determined)
^{a}Initial inoculation level: Hafinia alvei: 1.9 × 10⁸ CFU/ml; Klebsiella oxytoca: 2.1 × 10⁹ CFU/ml; Proteus hauseri: 1.3 × 10⁹ CFU/ml; Serratia marcesens: 1.2 × 10⁹ CFU/ml; Shigella flexneri: 1.1 × 10⁹ CFU/ml; Shigella sonnei: 1.3 × 10⁹ CFU/ml; Staphylococcus aureus: 1.9 × 10⁸ CFU/ml; Aerococcus virians: 1.0 × 10⁸ CFU/ml; Yersinia pseudotuberculosis: 1.0 × 10⁹ CFU/ml; E. coli O26:H11: 1.2 × 10⁹ CFU/ml; E. coli O111:NM: 1.1 × 10⁹; Vibro cholerae: 1.2 × 10⁶ CFU/ml; Campylobacter jejuni: 1.2 × 10¹⁰ CFU/ml.
^{b}The actual time 0 was delayed by 5 to 10 seconds due to time for sample processing.
^{c}ND, not determined.
^{d}Negative by direct plating and enrichment culture.

### Example 2

### Efficacy of the Organic Acid/SDS Compositions Against L. Monocytogenes

The efficacy of the antibacterial compositions disclosed herein was tested against Listeria monocytogenes using the same assay and procedures disclosed in Example 1. The results are indicated in Table 10.

TABLE 10
Reduction of L. monocytogenes by different concentrations of
levulinic acid and SDS individually and in combination at 21° C.

| | L. monocytogenes counts (log CFU/ml) at min: | | | | | |
|---|---|---|---|---|---|---|
| Chemical Treatment | 0^{a} | 2 | 5 | 10 | 20 | 30 |
| 0.5% levulinic acid (pH 3.1) | 6.7 ± 0.2 | 6.7 ± 0.1 | 6.8 ± 0.3 | 6.9 ± 0.2 | 6.7 ± 0.2 | 6.8 ± 0.2 |
| 1.0% levulinic acid (pH 3.0) | 6.8 ± 0.3 | 6.7 ± 0.2 | 6.6 ± 0.3 | 6.6 ± 0.3 | 6.6 ± 0.0 | 6.6 ± 0.3 |
| 1.5% levulinic acid (pH 2.9) | 6.9 ± 0.1 | 6.9 ± 0.2 | 6.9 ± 0.3 | 6.9 ± 0.1 | 6.9 ± 0.3 | 6.8 ± 0.3 |
| 2.0% levulinic acid (pH 2.9) | 6.8 ± 0.3 | 6.8 ± 0.2 | 6.9 ± 0.2 | 6.7 ± 0.2 | 6.9 ± 0.2 | 6.8 ± 0.2 |
| 0.05% sodium dodecyl | 6.6 ± 0.3 | 6.4 ± 0.1 | 6.0 ± 0.1 | 5.0 ± 0.3 | 3.8 ± 0.2 | 3.3 ± 0.1 |
| sulfate (pH 4.8) | | | | | | |
| 0.5% levulinic acid + | −^{c} | − | − | − | − | − |
| 0.05% SDS (pH 3.0) | | | | | | |

^{a}The actual time 0 was delayed by 5 to 10 seconds due to time for sample processing.
^{b}+, Positive by enrichment culture but not by direct plating (minimum detection level is 0.7 log CFU/ml).
^{c}−, Negative by direct plating and enrichment culture.

### Example 3

### Reduction of Microorganisms by Different Chemical Combination at 21° C.

Different combinations of pharmaceutically acceptable acids in combination with various pharmaceutically acceptable surfactants were tested for their antibacterial properties.

Microorganisms were contacted with the test compositions using the same assay and procedures as disclosed in Example 1. The results obtained by contacting microorganisms with different surfactant/acid combinations are indicated in Tables 9-13. Reduction of S. enteritidis and aerobic plate counts on ripen tomato by levulinic acid plus SDS treatment is presented in Table 12. As indicated by the following data, particularly Tables 12 & 13, not all organic acids/surfactant combinations perform equivalently with regards to their efficacy as antimicrobial agents.

TABLE 11
Reduction of microorganisms by different chemical combination at 21° C.

| Chemical treatment | 0^{a} | 1 | 2 | 5 | 10 | 20 | 30 | 60 |
|---|---|---|---|---|---|---|---|---|
| | E. coli O157:H7 counts (log CFU/ml) at min: | | | | | | | |
| E. coli O157:H7 only | 7.2 | 7.4 | ND^{b} | 7.3 | ND | ND | 7.3 | 7.4 |
| (Control) | | | | | | | | |
| 0.05% SDS to pH 3.0 | <0.7 | <0.7 | <0.7 | <0.7 | <0.7 | 0.7 | <0.7 | <0.7 |
| by 1 N HCl | | | | | | | | |
| | S. Enteritidis counts (log CFU/ml) at min: | | | | | | | |
| S. Enteritidis only | 7.2 | 7.1 | ND | 7.2 | ND | ND | 7.4 | 7.3 |
| (Control) | | | | | | | | |
| 0.05% SDS to pH 3.0 | <0.7 | <0.7 | <0.7 | <0.7 | <0.7 | <0.7 | <0.7 | <0.7 |
| by 1 N HCl | | | | | | | | |
| | Y. pestis counts (log CFU/ml) at min: | | | | | | | |
| Y. pestis only | 6.3 | 6.1 | 6.4 | 6.7 | 6.6 | 6.5 | 6.7 | 6.7 |
| (Control) | | | | | | | | |
| 0.5% Levulinic acid | <0.7 | <0.7 | <0.7 | <0.7 | <0.7 | <0.7 | <0.7 | <0.7 |
| plus 0.05% SDS | | | | | | | | |
| (pH 3.0) | | | | | | | | |

^{a}The actual time 0 was delayed by 5 to 10 seconds due to time for sample processing.
^{b}ND, not determined.

TABLE 12
Reduction of S. Enteritidis and aerobic plate counts on
ripen tomato by levulinic acid plus SDS treatment at 21° C.

| | | | | | In treatment |
|---|---|---|---|---|---|
| Treatment | 0^{a} | 1 | 2 | 5 | solution (5 min) |
| | S. Enteritidis counts (log CFU/g) at min: | | | | |
| PBS (7.2) (Control) | 5.0 | 4.7 | 4.7 | 4.9 | 5.6 |
| 0.5% levulinic acid + | 4.0 | 2.4 | 2.4 | 2.6 | +^{b} |
| 0.05% SDS (pH 3.1) | | | | | |
| | Aerobic plate counts (log CFU/g) at min: | | | | |
| PBS (pH 7.2) (Control) | 5.2 | 5.0 | 4.7 | 5.0 | 5.8 |
| 0.5% levulinic acid + | 4.7 | 3.1 | 3.1 | 3.0 | 1.0 |
| 0.05% SDS (pH 3.1) | | | | | |
| | S. Enteritidis counts (log CFU/g) at min: | | | | |
| PBS (7.2) (Control) | 5.8 | 5.5 | 5.2 | 5.1 | 5.9 |
| 1.0% levulinic acid + | 5.3 | 2.9 | 2.9 | 1.8 | + |
| 0.1% SDS (pH 2.8) | | | | | |
| | Aerobic plate counts (log CFU/g) at min: | | | | |
| PBS (pH 7.2) (Control) | 5.9 | 5.6 | 5.4 | 5.1 | 6.0 |
| 1.0% levulinic acid + | 5.5 | 3.1 | 3.1 | 2.1 | 3.1 |
| 0.1% SDS (pH 2.8) | | | | | |
| | S. Enteritidis counts (log CFU/g) at min: | | | | |
| PBS (7.2) (Control) | 5.8 | 5.5 | 5.2 | 5.1 | 5.9 |
| 2.0% levulinic acid + | 4.4 | 1.9 | + | + | + |
| 1.0% SDS (pH 2.7) | | | | | |
| | Aerobic plate counts (log CFU/g) at min: | | | | |
| PBS (pH 7.2) (Control) | 5.9 | 5.6 | 5.4 | 5.1 | 6.0 |
| 2.0% levulinic acid + | 4.7 | 2.3 | 1.0 | 1.1 | 1.8 |
| 1.0% SDS (pH 2.7) | | | | | |

^{a}The actual time 0 may was delayed by 10 to 20 seconds due to time for sample processing.
^{b}+, Below the minimum detection level by direct plating (<0.7 log CFU/ml), but positive by enrichment culture.

TABLE 13
Reduction of E. coli O157:H7 by combination of different acids and SDS at 21° C.

| | Bacterial counts (log CFU/ml) at min: | | | | | | | |
|---|---|---|---|---|---|---|---|---|
| Chemical treatment | 0^{a} | 1 | 2 | 5 | 10 | 20 | 30 | 60 |
| E. coli O157:H7 only (Control) | 7.7 | 7.6 | 7.7 | 7.7 | 7.8 | 7.7 | 7.8 | 7.7 |
| 0.5% adipic acid plus 0.05% | 2.7 | 1.7 | +^{b} | −^{c} | − | − | − | − |
| benzalkonium chloride (pH 3.1) | | | | | | | | |
| 0.5% cetylpyidinum chloride | + | + | + | + | + | + | + | + |
| plus 0.05% SDS (pH 5.8) | | | | | | | | |
| 0.5% citric acid plus 0.05% | + | + | − | − | − | − | − | − |
| SDS (pH 2.5) | | | | | | | | |
| 0.5% ethylenediaminetetraacetic | − | − | − | − | − | − | − | − |
| acid plus 0.05% SDS (pH 3.0) | | | | | | | | |
| 0.5% eugenol plus 0.05% SDS | − | − | − | − | − | − | − | − |
| (pH 2.6) | | | | | | | | |
| 0.5% Fumaric acid plus 0.05% | + | − | − | − | − | − | − | − |
| SDS (pH 2.4) | | | | | | | | |
| 0.5% hexanoic acid plus 0.05% | 2.7 | 1.7 | − | − | − | − | − | − |
| SDS (pH 3.2) | | | | | | | | |
| 0.5% levulinic acid plus 0.05% | − | − | − | − | − | − | − | − |
| benzalkonium chloride (pH 3.1) | | | | | | | | |
| 0.5% levulinic acid plus 0.05% | − | − | − | − | − | − | − | − |
| cetypridinium chloride (pH 3.1) | | | | | | | | |
| 0.5% levulinic acid plus 0.05% | >5.3 | >5.3 | >5.3 | >5.3 | >5.3 | >5.3 | >5.3 | >5.3 |
| cocamide MEA (pH 3.1) | | | | | | | | |
| 0.5% malic acid plus 0.05% | + | + | − | − | − | − | − | − |
| SDS (pH 2.6) | | | | | | | | |
| 0.5% phosphoric acid plus | − | − | − | − | − | − | − | − |
| 0.05% SDS (pH 1.7) | | | | | | | | |
| 0.5% succinic acid plus 0.05% | − | − | − | − | − | − | − | − |
| SDS (pH 2.9) | | | | | | | | |
| 0.5% tartaric acid plus 0.05% | + | + | + | − | − | − | − | − |
| SDS (pH 2.5) | | | | | | | | |

^{a}The actual time 0 was delayed by 5 to 10 seconds due to time for sample processing.
^{b}+, Positive by enrichment culture but not by direct plating (minimum detection level is 0.7 log CFU/ml).
^{c}−, Negative by both direct plating and enrichment culture.

TABLE 14
Reduction of S. Enteritidis by combination of different acids and SDS at 21° C.

| | Bacterial counts (log CFU/ml) at min: | | | | | | | |
|---|---|---|---|---|---|---|---|---|
| Chemical treatment | 0^{a} | 1 | 2 | 5 | 10 | 20 | 30 | 60 |
| S. Enteritidis only (Control) | 7.5 | 7.6 | 7.4 | 7.6 | 7.5 | 7.4 | 7.6 | 7.5 |
| 0.5% adipic acid plus 0.05% | +^{b} | −^{c} | − | − | − | − | − | − |
| benzalkonium chloride (pH 3.1) | | | | | | | | |
| 0.5% cetylpyidinum chloride | + | + | + | + | + | + | + | + |
| plus 0.05% SDS (pH 5.8) | | | | | | | | |
| 0.5% citric acid plus 0.05% | + | + | − | − | − | − | − | − |
| SDS (pH 2.5) | | | | | | | | |
| 0.5% ethylenediaminetetraacetic | + | + | + | − | − | − | − | − |
| acid plus 0.05% SDS (pH 3.0) | | | | | | | | |
| 0.5% eugenol plus 0.05% SDS | − | − | − | − | − | − | − | − |
| (pH 2.6) | | | | | | | | |
| 0.5% Fumaric acid plus 0.05% | + | + | + | − | − | − | − | − |
| SDS (pH 2.4) | | | | | | | | |
| 0.5% hexanoic acid plus 0.05% | − | − | − | − | − | − | − | − |
| SDS (pH 3.2) | | | | | | | | |
| 0.5% levulinic acid plus 0.05% | − | − | − | − | − | − | − | − |
| benzalkonium chloride (pH 3.1) | | | | | | | | |
| 0.5% levulinic acid plus 0.05% | − | − | − | − | − | − | − | − |
| cetypridinium chloride (pH 3.1) | | | | | | | | |
| 0.5% levulinic acid plus 0.05% | >5.8 | >5.8 | >5.8 | >5.8 | >5.8 | 5.3 | 4.8 | 4.1 |
| cocamide MEA (pH 3.1) | | | | | | | | |
| 0.5% malic acid plus 0.05% | + | + | − | − | − | − | − | − |
| SDS (pH 2.6) | | | | | | | | |
| 0.5% phosphoric acid plus | + | − | − | − | − | − | − | − |
| 0.05% SDS (pH 1.7) | | | | | | | | |
| 0.5% succinic acid plus 0.05% | + | − | − | − | − | − | − | − |
| SDS (pH 2.9) | | | | | | | | |
| 0.5% tartaric acid plus 0.05% | + | + | + | − | − | − | − | − |
| SDS (pH 2.5) | | | | | | | | |

^{a}The actual time 0 was delayed by 5 to 10 seconds due to time for sample processing.
^{b}+, Positive by enrichment culture but not by direct plating (minimum detection level is 0.7 log CFU/ml).
^{c}−, Negative by both direct plating and enrichment culture.

The results shown in Tables 10-12 are of special relevance as they indicate the superiority of certain acid/surfactant combinations over others. More particularly, Tables 10-12 indicate that the choice of acid leads to different antimicrobial activity even when different acids are used in the same concentrations. These tables also indicate that the number of protic hydrogen atoms in a given acid is not relevant to the bactericidal activity of the solutions, as illustrated by the different activities observed when H₄EDTA (a tetraprotic acid), citric acid (a triprotic) or fumaric acid (a diprotic acid) are used instead linear-chain mono-acids. This may be due to the smaller acidity of small, multiprotic acids vis-à-vis monoprotic ones, due to inter- and intra-molecular hydrogen bonding and also, in the case of H₄EDTA, of intramolecular transfer to two H⁺ moieties from the carboxyl group to the amine nitrogen.

Taken together, the results described in Tables 10-12 clearly indicate that not all acid/surfactant combinations display optimal antibacterial activity, when both activity time and intensity of the microbicidal effect are considered. Those tables suggest that linear, long (>4 carbon atoms) chain, monoprotic acids are preferred over others and ionic, long-chain surfactants (SDS, benzalkonium chloride, and cetylpyridinium chloride) are preferred over non-ionic surfactants (e.g., cocamide MEA).

Chain length may also be relevant as free-standing long-chains are more likely to disrupt cell walls. In the case of multiprotic acids, intramolecular hydrogen bonding may—at least in part—restrain these chains into a locked configuration, less likely to be disruptive of the lipid layer in cell walls. This is in part supported by the greater length of time needed for multiprotic acids to exhibit a measurable effect when compared with linear monoprotic acids.

### Example 4

### Efficacy of Compositions to Treat Contaminated Seeds

Since 1994, raw sprouts have been implicated as vehicles of outbreaks of E. coli O157:H7 and Salmonella both nationally and internationally. Most outbreaks were associated with alfalfa sprouts, but cress, mung bean, and clover sprouts have been implicated. Many treatments, including the use of heat and/or chemicals (e.g., NaOCl, Ca(OCl)₂, acidified NaClO₂, LiOCl, detergents, acidified ClO₂, Na₃PO₄, acidic calcium sulfate, and H₂O₂) have been evaluated for their ability to reduce E. coli O157:H7 contamination on alfalfa seeds. However none of these treatments can definitely eliminate the pathogen and render seeds with acceptable germination rates. Accordingly, applicants have investigated the ability of monoprotic acids/surfactant compositions as a wash solutions for eliminating E. coli O157:H7 and Salmonella from seeds, while retaining acceptable germination rates.

A 5-strain mixture of E. coli O157:H7 or S. Typhimurium at 10⁸ CFU/g was inoculated on alfalfa seeds. The seeds were dried at 21° C. for up to 72 h. A 0.5% levulinic acid and 0.05% SDS treatment for 5 min at 21° C. reduced E. coli O157:H7 and S. Typhimurium populations to undetectable levels (<5 CFU/g), however, some treated seeds were pathogen-positive by selective enrichment culture.

### Materials and Methods

Bacterial strains. To facilitate enumeration of E. coli O157:H7, nalidixic acid-resistant (50 μg/ml) strains were used. Five isolates of Escherichia coli O157:H7, including 932 (human isolate), E009 (beef isolate), E0018 (cattle isolate), E0122 (cattle isolate), E0139 (deer jerky isolate) or five isolates of Salmonella Typhmurium DT104, including H2662 (cattle isolate), 11942A (cattle isolate), 13068A (cattle isolate), 152N17-1 (dairy isolate) and H3279 (human isolate) were used as 5-strain composite mixtures.

Chemicals and chemical treatments. Levulinic acid at 0.5% and 0.05% and sodium dodecyl sulfate (SDS) were tested in combination at 21±2° C. as a wash treatment for their killing effect on E. coli O157:H7 and S. Typhimurium on alfalfa seeds. Calcium hypochlorite [20,000 μg/ml (ppm)] was used as a positive control and deionized water was used as a negative control.

Water. Deionized, unchlorinated water (filter sterilized through a 0.2-μm regenerated cellulose filter), tap water and autoclaved tap water were used.

Inoculation of alfalfa seeds. Alfalfa seeds were obtained from Caudill Seeds Co., Louisville, Ky., and had a germination rate of approximately 91%. Dry seeds (50 g) were placed in a sterilized glass beaker (1 L) and 5 ml of a 5-strain mixture of E. coli O157:H7 or S. Typhimurium DT 104 (10⁸-10⁹ CFU/ml or 10³-10⁴ CFU/ml) was inoculated on the surface of the seeds then dried in a laminar flow hood for 1, 4, 24, 48, and 72 h.

Determination of Salmonella and E. coli O157:H7 inactivation on alfalfa seeds. Inoculated and dried alfalfa seeds (50-g samples) were placed in a 1000-ml glass beakers containing 200 ml of levulinic acid plus SDS or controls and agitated at 150 rpm with a magnetic stir bar at 21° C. for 0, 1, 2, 5, 10, 20, 30 and 60 min Following treatment, the sample (1 or 25/g or ml) was placed in a stomacher bag containing 9 ml or 25 ml of 0.1 M phosphate buffer, pH 7.2 (PBS), or neutralizing buffer and pummeled for 1 minute at 150 rpm in a stomacher blender. The suspension was serially (1:10) diluted in 0.1% peptone water and 0.1 ml of each dilution was surface-plated in duplicate onto plates of TSA and Sorbitol MacConkey agar each containing 50 μg nalidixic acid/ml (TSA-NA and SMA-NA) for E. coli O157:H7; and TSA and XLD containing ampicillin (32 μg/ml), tetracycline (16 μg/ml) and streptomycin (64 μg/ml) (TSA+ and XLD+) for S. Typhimurium DT 104. All plates were incubated at 37° C. for 48 h.

Determination of seed germination percentage. To determine the germination percentage, treated and control seeds (5 gram per replicate) were placed on the surface of a plastic tray. A second tray containing 200 ml of sterile deionized water was placed with tray with seeds and water dropped into lower tray to maintain uniform moisture. The seeds were incubated at approximately 22° C. for 72 h.

### Results and Discussions

Results revealed that a viable population of 10⁸ CFU E. coli O157:H7/g of alfalfa seeds was present after drying for 4 h (Table 15). Treatments with 20,000 ppm calcium hypochlorite or 0.5% levulinic acid plus 0.05% SDS for up to 60 min reduced the E. coli O157:H7 population by greater than 6 and 5 log CFU/g, respectively.

The population of E. coli O157:H7 was reduced by 3 log CFU/g after drying for 24 h. Treatment with calcium hypochlorite and 0.5% levulinic acid plus 0.05% SDS for 5 min reduced E. coli O157:H7 populations to levels only detectable by enrichment culture. Similar results were observed with seeds dried for 48 and 72 h (Table 15).

Results revealed that a viable population of 10⁶ to 10⁷ CFU S. Typhimurium DT 104/g of alfalfa seeds was present after drying for 4 h. Treatments with 20,000 ppm calcium hypochlorite or 0.5% levulinic acid plus 0.05% SDS provided similar results, inactivating all Salmonella, including by enrichment culture, within 5 min (Table 16).

Drying seeds for 24, 48, or 72 h reduced the population of Salmonella by ca. 4 log CFU/g. Treatment with 20,000 ppm calcium hypochlorite or 0.5% levulinic acid plus 0.05% SDS for 5 min reduced Salmonella to levels undetectable by direct plating, but still detectable by enrichment culture (Table 16).

TABLE 15
Counts of E. coli O157:H7 on alfalfa seeds initially
inoculated with 10⁸ CFU/g and dried at 21° C.
in a laminar hood for different periods of time

| | Min of exposure | | | | | | | |
|---|---|---|---|---|---|---|---|---|
| Treatment method | 0^{a} | 1 | 2 | 5 | 10 | 20 | 30 | 60 |
| | E. coli O157:H7 counts (CFU/g) | | | | | | | |
| | on seeds dried for 4 h | | | | | | | |
| 0.1 M PBS, pH 7.2 | 8.1 | 8.2 | 8.2 | 8.1 | 8.2 | 8.3 | 8.3 | 8.1 |
| 20,000 ppm, | +^{b} | + | −^{c} | + | 1.7 | 2.0 | 1.7 | − |
| Ca(OCl)₂, pH 11.4 | | | | | | | | |
| 0.5% levulinic | 1.7 | 2.7 | 3.0 | 2.5 | 2.8 | 2.0 | 2.6 | 2.2 |
| acid + 0.05% | | | | | | | | |
| SDS, pH 3.2 | | | | | | | | |
| | E. coli O157:H7 counts (CFU/g) | | | | | | | |
| | on seeds dried for 24 h | | | | | | | |
| 0.1 M PBS, pH 7.2 | 4.7 | 4.8 | 4.9 | 5.0 | 4.7 | 4.9 | 4.8 | 4.9 |
| 20,000 ppm, | + | − | − | − | + | − | − | + |
| Ca(OCl)₂, pH 11.4 | | | | | | | | |
| 0.5% levulinic | 1.7 | 1.4 | 0.7 | + | + | + | − | + |
| acid + 0.05% | | | | | | | | |
| SDS, pH 3.2 | | | | | | | | |
| | E. coli 0157:H7 counts (CFU/g) | | | | | | | |
| | on seeds dried for 48 h | | | | | | | |
| 0.1 M PBS, pH 7.2 | 4.0 | 4.1 | 4.0 | 4.1 | 4.1 | 4.0 | 4.0 | 3.9 |
| 20,000 ppm, | + | − | + | − | − | − | − | − |
| Ca(OCl)₂, pH 11.4 | | | | | | | | |
| 0.5% levulinic | 2.7 | 2.1 | + | + | + | + | + | + |
| acid + 0.05% | | | | | | | | |
| SDS, pH 3.2 | | | | | | | | |
| | E. coli 0157:H7 counts (CFU/g) | | | | | | | |
| | on seeds dried for 72 h | | | | | | | |
| 0.1 M PBS, pH 7.2 | 3.8 | 3.9 | 3.9 | 4.0 | 4.0 | 4.1 | 4.0 | 4.1 |
| 20,000 ppm, | + | + | + | − | + | + | − | − |
| Ca(OCl)₂, pH 11.4 | | | | | | | | |
| 0.5% levulinic | 1.9 | 1.4 | 1.1 | + | + | − | − | + |
| acid + 0.05% | | | | | | | | |
| SDS, pH 3.2 | | | | | | | | |

^{a}The actual time 0 was delayed by 20 to 30 seconds due to time for sample processing.
^{b}+, Below the minimum detection level by direct plating (<1.7 log CFU/ml), but positive by enrichment culture.
^{c}−, Negative by direct plating and enrichment culture.

TABLE 16
Counts of S. Typhimurium DT 104 on alfalfa seeds
initially inoculated with 10⁸ CFU/g and dried at 21°
C. in a laminar hood for different periods of time

| | Min of exposure | | | | | | | |
|---|---|---|---|---|---|---|---|---|
| Treatment method | 0^{a} | 1 | 2 | 5 | 10 | 20 | 30 | 60 |
| | S. Typhimurium DT 104 counts (CFU/g) | | | | | | | |
| | in seeds dried for 4 h | | | | | | | |
| 0.1 M PBS, pH 7.2 | 6.4 | 6.8 | 6.3 | 6.4 | 6.6 | 6.3 | 6.3 | 6.0 |
| 20,000 ppm, | +^{b} | −^{c} | − | − | − | − | − | − |
| Ca(OCl)₂, pH 11.4 | | | | | | | | |
| 0.5% levulinic | 3.1 | + | + | − | − | − | − | − |
| acid + 0.05% | | | | | | | | |
| SDS, pH 3.2 | | | | | | | | |
| | S. Typhimurium DT 104 counts (CFU/g) | | | | | | | |
| | in seeds dried for 24 h | | | | | | | |
| 0.1 M PBS, pH 7.2 | 4.4 | 4.2 | 4.3 | 4.4 | 4.5 | 4.6 | 4.6 | 4.3 |
| 20,000 ppm, | + | + | − | − | − | − | − | − |
| Ca(OCl)₂, pH 11.4 | | | | | | | | |
| 0.5% levulinic | 1.6 | 2.4 | 1.2 | + | + | − | − | − |
| acid + 0.05% | | | | | | | | |
| SDS, pH 3.2 | | | | | | | | |
| | S. Typhimurium DT 104 counts (CFU/g) | | | | | | | |
| | in seeds dried for 48 | | | | | | | |
| 0.1 M PBS, pH 7.2 | 4.0 | 4.1 | 4.2 | 4.3 | 4.2 | 4.4 | 4.4 | 4.3 |
| 20,000 ppm, | + | + | − | − | + | − | − | − |
| Ca(OCl)₂, pH 11.4 | | | | | | | | |
| 0.5% levulinic | 3.0 | + | − | − | + | − | + | − |
| acid + 0.05% | | | | | | | | |
| SDS, pH 3.2 | | | | | | | | |
| | S. Typhimurium DT 104 counts (CFU/g) | | | | | | | |
| | in seeds dried for 72 | | | | | | | |
| 0.1 M PBS, pH 7.2 | 4.0 | 4.0 | 3.9 | 4.1 | 4.1 | 4.5 | 4.1 | 4.2 |
| 20,000 ppm, | − | − | − | − | − | + | + | − |
| Ca(OCl)2, pH 11.4 | | | | | | | | |
| 0.5% levulinic | 2.3 | + | + | + | − | − | + | + |
| acid + 0.05% | | | | | | | | |
| SDS, pH 3.2 | | | | | | | | |

^{a}The actual time 0 was delayed by 20 to 30 seconds due to time for sample processing.
^{b}+, Below the minimum detection level by direct plating (<1.7 log CFU/ml), but positive by enrichment culture.
^{c}−, Negative by direct plating and enrichment culture.

Both chemical treatment solutions were negative for E. coli O157:H7 or Salmonella following treatment of contaminated seeds. Seeds treated for 10 min were transferred to a stomacher bag and pummeled for another 10 min at 200 rpm. Results revealed that all five samples treated with 20,000 ppm calcium hypochlorite or 0.5% levulinic acid and 0.05% SDS were E. coli O157:H7- and Salmonella-negative by direct plating, whereas (two of ten samples) treated with 0.5% levulinic acid and 0.05% SDS were negative by enrichment culture.

The germination rate of alfalfa seed treated with 0.5% levulinic acid plus 0.05% SDS for 1 hour at 21° C. was 80%, with tap water was 71%, and for 20,000 ppm calcium hypochlorite was 47.3%.

### Conclusion

Similar results of E. coli O157:H7 and Salmonella inactivation on alfalfa seeds were obtained with treatments of 20,000 ppm calcium hypochlorite, pH 11.4, or 0.5% levulinic acid plus 0.05% SDS, pH 3.2. Alfalfa seed germination percentages were substantially greater when treated with levulinic acid plus SDS relative to treatments using calcium hypochlorite.

### Example 5

### The Determination of Shelf-Life of Treated Lettuce

Whole Romaine lettuce (3 heads in each bag) was soaked in a plastic container with 5 liters of solution composed of 0.5% levulinic acid plus 0.05% SDS, pH 2.9 at 21° C. for either 15 or 30 min, then rinsed in same amount of tap water for 3 times. The samples of treated lettuce (inner and outer leaves) were kept in a layer of paper towel and dried in a laminar hood for 30 min for removing extra water. Then, the lettuce was kept in the original bag at 5° C. The lettuce treated with tap water only was used as the negative control.

Results indicated that the color, shape, and fragility of lettuce treated with 0.5% levulinic acid plus 0.05% SDS for either 15 or 30 min was the same in 20 days when compared with lettuce treated with water only. At 30 days, these characteristics, including color, shape, and fragility were better when compared with lettuce treated with water, in which showed some decays in bacteria—and/or fungi—induced decay of the surface of lettuce.

### Example 6

### Reduction of E. coli O157:H7 and Salmonella in Ground Beef

The goal of this experiment is to develop and validate a practical treatment to eliminate/reduce E. coli O157:H7 and Salmonella contamination in ground beef. As disclosed in Example 1 the combination of 0.5% levulinic acid and 0.05% SDS inactivates E. coli O157:H7, Salmonella Enteritidis, and S. Typhimurium DT 104 (>10⁷ CFU/ml) within 10 sec (processing time) when tested in pure culture. Treatment of lettuce with a combination of 3% levulinic acid plus 1% SDS, pH 2.7, for <20 sec reduced both Salmonella and E. coli O157:H7 cell numbers by >6.7 log CFU/g. Salmonella and aerobic bacteria cell numbers on chicken wings were reduced by >5 log CFU/g by treatment with 3% levulinic acid plus 2% SDS, pH 2.7, for 1 min. However, levulinic acid at 0.5% and SDS at 0.05% have relatively little bactericidal activity when they are used individually.

Phase 1 of the experiments will determine the relationship between different chemical concentrations and rinse exposure time at 5° C. on inactivation of E. coli O157:H7 or Salmonella on beef trim pieces. A 5-strain mixture of E. coli O157:H7 or Salmonella, including Typhimurium DT 104, will be used. Beef trim will be cut into ca. 2-in cubes. Two inoculation levels (high inoculum at 10⁵ CFU/g and low inoculum at 10² CFU/g) will be used. Following inoculation, the meat pieces (45 in each group) will be held at 5° C. for 1, 2, 4, 24 h for pathogen attachment and acclimation. Three treatment methods (levulinic acid+SDS, acidified sodium chlorite, and water only) will be compared for antimicrobial activity. The concentration of levulinic acid will range from 0.5 to 3.0% and of SDS from 0.05 to 2.0% and treatments will be applied at 5° C. for 1, 2, 3, 4, and 5 min. Each meat piece will be treated in a stomacher bag then removed to another bag containing 0.1 M phosphate-buffer or neutralizing buffer to stop further chemical activity. All treatment and washing solutions will be assayed for either E. coli O157:H7 or Salmonella and aerobic plate counts (APC).

Phase 2 of the experiment will evaluate whether E. coli O157:H7 or Salmonella can be recovered from ground beef prepared from levulinic acid+SDS-treated beef trim and stored frozen for up to 6 months. The concentration of levulinic acid+SDS and exposure time at 5° C. to be used will be based on the data obtained from Phase 1 studies. Beef trim treated by the three methods described for the Phase 1 study will be ground, formed into patties, packaged and frozen at −20° C. for up to 6 months. Beef patties will be assayed monthly for either E. coli O157:H7 or Salmonella and APC.

Phase 3 of the experiment will validate the best levulinic acid and SDS concentrations and exposure time to treat beef trim and confirm under storage conditions inactivation of E. coli O157:H7 and Salmonella in ground beef made from the treated beef trim. Beef will be cut into ca. 2-in cubes and a volume of 1.0-ml of bacterial solution containing ca. 10,000 CFU E. coli O157:H7 or S. Typhimurium DT 104 will be inoculated on the surface. The beef cubes will be mixed, held at 5° C. for 3 h and then treated with levulinic acid and SDS at concentrations and an exposure time determined in Phase 1 and 2 studies. After treatment, the beef cubes will be ground as a mixture. The ground meat will be packaged, frozen, stored at −20° C. for up to 3 months, and assayed periodically for E. coli O157:H7 or Salmonella and APC.

Many pathogen reduction interventions in the meat and poultry industry involve the use of acids or antimicrobial chemical treatments, but most of these interventions reduce E. coli O157:H7 or Salmonella contamination by only 10- to 100-fold. There were in 2007 22 recalls of ground beef contaminated with E. coli O157:H7, indicating there are opportunities for more effective antimicrobial interventions in the meat industry. The levulinic acid plus SDS treatment disclosed herein can greatly reduce by >5 log CFU/g E. coli O157:H7 and Salmonella contamination of produce and poultry and may also be useful for beef. In addition the shelf life of treated meat may be extended because of reduction of spoilage bacteria. Levulinic acid was selected as the primary focus of this study because it can be produced at low cost and in high yield from renewable feedstocks. Its safety for human application through respiratory absorption has been widely tested and it has GRAS status for direct addition to food as a favoring substance or adjunct (24, FDA 2008, 21 CFR, 172.515). Sodium dodecyl sulfate has GRAS status for multipurpose additives (25, FDA 2007, 21 CFR, 172.822). It is approved for use in a variety of foods, including egg whites, fruit juices, vegetable oils, and gelatin as a whipping or as a wetting agent.

### Example 7

### Treatment of Biofilms with Compositions Comprising an Acid and Surfactant

### Materials and Methods

Preparation of Stainless Steel Coupons.

Coupons (4 cm×2.5 cm) composed of different materials, including stainless steel, polyvinyl chloride, nitrile rubber, glass, ultra-high molecular weight polyethylene were washed by a 10-min immersion with agitation (150 rpm) in 1000 ml of an aqueous 2% RBS 35 Detergent Concentrate solution (20 ml of RBS 35 Concentrate per liter of tap water at 50° C.; Pierce, Rockford, Ill.), and rinsed by immersion in 1000 ml of tap water (initial at 50° C.) with agitation (150 rpm) for 25 min. Five additional 1-min immersions with agitation (150 rpm) in 1000 ml of distilled water at ambient temperature were performed. The coupons were dried. The coupons were then individually wrapped and autoclaved at 121° C. for 30 min.

Biofilm formation of S. Enteritidis on coupons:

For purpose of a well-formatted biofilm of S. Enteritidis on the surface of coupons, the coupons were placed individually in a 250-ml flask containing 100 ml tryptic soy broth (TSB) and an inoculum of 1.0 ml ca. 10⁸ CFU of a 5-strain mixture of S. Enteritidis was added. The flasks were incubated at 37° C. for 24 h. The coupons then were removed individually and placed on the surface of a layer of paper tower for absorbing the extra fluid of the surface.

The coupons having the formed biofilms were then individually transferred to plates containing 30 ml chemical solution for treatment for 0, 1, 2, 5, 10, 20 min. Following treatment each coupon was placed in a 50 ml centrifuge tube containing 9.0 ml of PBS and 30 glass beads (5 mm). The tubes were agitated by a Vortex for 2 min to suspend the adherent bacteria. The suspended bacteria were serially diluted (1:10) in 0.1% peptone and plated in duplicate on TSA and XLD agar plates for S. Enteritidis enumeration. The plates were incubated for 48 h at 37° C. and bacterial colonies counted.

### Results

Studies of S. Enteritidis attached to the surface of the coupons revealed that the pathogen was eliminated in less than 1 minute by the treatment solution containing 3% levulinic acid plus 2% SDS (Tables 16 & 17).

Furthermore, studies using (concentration 3% levulinic acid plus 2% SDS) showed that these solutions when sprayed onto the surface of the coupons, led to the development of a persistent (>20 minutes if left undisturbed) antibacterial foam that prolonged the activity and efficacy of the invention against bacterial films. Said foam also aids in the removal, by flotation, of particulate material from the treated surface.

TABLE 17
Reduction of S. Enteritidis on stainless steel coupons
by levulinic acid plus SDS treatment at 21° C.

| Treatment | 0^{a} | 1 | 2 | 5 | 10 | 20 |
|---|---|---|---|---|---|---|
| | S. Enteritidis counts (log CFU/cm²) | | | | | |
| | with coupons incubated for 2 h at min: | | | | | |
| PBS (7.2) (Control) | 7.4 | 7.3 | ND^{b} | 7.3 | ND | 7.4 |
| 3% levulinic acid + | <0.7 | <0.7 | <0.7 | <0.7 | <0.7 | <0.7 |
| 2% SDS (pH 2.7) | | | | | | |
| | S. Enteritidis counts (log CFU/cm²) | | | | | |
| | with coupons incubated for 4 h at min: | | | | | |
| | <0.7 | <0.7 | <0.7 | <0.7 | <0.7 | <0.7 |
| | S. Enteritidis counts (log CFU/cm²) | | | | | |
| | with coupons incubated for 24 h at min: | | | | | |
| | <0.7 | <0.7 | <0.7 | <0.7 | <0.7 | <0.7 |

TABLE 18
Chemical inactivation of S. Enteritidis in biofilm at 21°
C. by 3% levulinic acid plus 2% sodium dodecyl sulfate (SDS)

| | | S. Enteritidis count | | | |
|---|---|---|---|---|---|
| Type of | | (log CFU/cm2) at minutes | | | |
| Coupon | Treatment Solution | 0 | 1 | 5 | 10 |
| Stainless | PBS, pH 7.2 | 8.0 | 8.4 | 8.6 | 8.2 |
| | NaClO₂ (500 ppm), pH 2.8 | 7.5 | 5.9 | 5.4 | 6.2 |
| | 3.0% levulinic acid (LV) | <0.7 | <0.7 | <0.7 | <0.7 |
| | plus 2.0% SDS, pH 3.0 | | | | |
| Polyvinyl | PBS | 8.8 | 9.0 | 8.8 | 8.0 |
| chloride | NaClO₂ (500 ppm) | 6.9 | 5.5 | 5.3 | 4.2 |
| | 3.0% LV plus 2.0% SDS | 2.3 | 1.7 | 2.2 | <0.7 |
| Nitrile | PBS | 7.8 | 8.0 | 7.7 | 7.9 |
| rubber | NaClO₂ (500 ppm) | 7.2 | 5.2 | 2.6 | 1.3 |
| | 3.0% LV plus 2.0% SDS | 4.1 | 1.7 | <0.7 | <0.7 |
| Glass | PBS | 8.2 | 8.7 | 8.4 | 8.4 |
| | NaClO₂ (500 ppm) | 6.8 | 3.3 | <0.7 | <0.7 |
| | 3.0% LV plus 2.0% SDS | <0.7 | <0.7 | <0.7 | <0.7 |
| Ultra-high | PBS | 8.4 | 8.4 | 8.4 | 8.4 |
| molecular | NaClO2 (500 ppm) | 6.8 | 6.1 | <0.7 | <0.7 |
| weight | 3.0% LV plus 2.0% SDS | <0.7 | <0.7 | <0.7 | <0.7 |
| polyethylene | | | | | |

TABLE 19
Inactivation of Salmonella Enteritidis in biofilm at 21°
C. by foamed 3% levulinic acid plus 2% sodium dodecyl sulfate (SDS)

| | | Counting of Salmonella Enteritidis | | | | | |
|---|---|---|---|---|---|---|---|
| | | (log₁₀ CFU/cm²) at minutes | | | | | |
| Coupon type | Chemical solution | 0^{a} | 1 | 2 | 5 | 10 | 20 |
| Stainless | PBS, pH 7.2 | 7.3 | 7.7 | 8.0 | 7.2 | 8.0 | 7.3 |
| | 3% levulinic acid plus | 8.3 | 6.7 | 6.8 | 4.0 | 2.3 | 2.0 |
| | 2% SDS, pH 2.8 | | | | | | |
| Polyvinyl | PBS, pH 7.2 | 8.0 | 8.3 | 8.2 | 8.6 | 8.2 | 8.6 |
| chloride | 3% levulinic acid plus | 5.8 | 4.9 | 3.1 | 3.0 | 3.2 | 1.0 |
| | 2% SDS, pH 2.8 | | | | | | |
| Nitrile | PBS, pH 7.2 | 7.4 | 7.6 | 7.6 | 7.5 | 7.4 | 7.2 |
| rubber | 3% levulinic acid plus | 7.1 | 4.1 | 3.5 | 3.3 | 2.4 | 1.7 |
| | 2% SDS pH 2.8 | | | | | | |
| Glass | PBS, pH 7.2 | 8.0 | 8.5 | 7.7 | 7.9 | 7.8 | 7.9 |
| | 3% levulinic acid plus | 4.9 | 4.4 | 3.3 | 3.5 | 1.7 | 1.7 |
| | 2% SDS, pH 2.8 | | | | | | |
| Ultra-high | PBS, pH 7.2 | 6.9 | 6.9 | 6.7 | 6.4 | 6.7 | 6.3 |
| molecular | 3% levulinic acid plus | 5.4 | 4.6 | 2.9 | 2.3 | 1.7 | 1.7 |
| weight | 2% SDS | | | | | | |
| polyethylene | | | | | | | |

^{a}The actual time 0 was delayed by 35 to 45 seconds due to time for sample processing.

Work by (Wang, H., H. Liang, Y. Luo, and V. Malyarchuk, 2009. “Effect of surface roughness on retention and removal of Escherichia coli O157:H7 on surfaces of selected fruits”, J. Food Sciences, 74:E8-E15) using confocal laser scanning microscopy analysis of fruits has demonstrated surface roughness (Rₐ) of fruits allows bacteria to attach into surface grooves thus making removal of such bacteria more difficult. Among the four fruits tested by Wang, et al., including Golden Delicious apples, navel oranges, avocados, and cantaloupes, apples had the smoothest surface, while the cantaloupes had the highest Rₐ value. Rough and irregular fruit surfaces were found not only to provide a safe harbor for foodborne pathogens, such fruits when treated with peroxyacetic acid, acidic electrolyzed water or deionized water actually showed an increase in the adhesion rates of E. coli on these rough surfaces. Applicants anticipate that since the levulinic acid comprising compositions of the present invention have shown effectiveness against biofilms, the compositions are also anticipated to be unique in their ability to remove pathogens from rough surfaced food substances while retaining the organoleptic properties of the food. Indeed, Applicants' previous results involving leafy vegetables with varied roughness, and with both ground beef and poultry meat—all substrates that provide cavities and grooves appropriate to foment bacterial growth, support such anticipated efficacy of the presently disclosed antimicrobial compositions for treating rough fruits to remove pathogenic organisms.

### Example 8

### Efficacy of Compositions to Kill Spores of Bacillus anthracis Sterne

### Methods:

For all experiments an equal volume of spore suspension of B. anthracis Sterne (34F₂) was added to 25 ml of reagents A, B, C, D, E, and F in 250-ml flasks. The compositions of reagents are as follows

A. 3% levulinic acid plus 2% SDS,

B. 2% levulinic acid plus 1% SDS,

C. 0.5% levulinic acid plus 0.05% SDS,

D. 3% levulinic acid,

E. 2% SDS,

F. water (serving as the control)

Flasks were incubated at 37° C. in a shaker (200 rpm). At each time point 100 μl of sample was transferred into 900 μl water, vortexed, and 100 μl of the dilution spread on Brain Heart Infusion agar plates. Plates were incubated at 37° C. over night and colonies counted the next morning (approximately 16 hours later).

### Experiment A3:

250 μl spore suspension (5×10⁴ spores) were added to 25 ml of the reagents. Sampling time points were t0 (spores were added and after mixing with the reagent, 100 μl of the suspension were removed for enumeration), t10 min, t45 min, t90 min, t180 min. Average plate counts (FIGS. 18A-18E) are based on counting three plates; error bars indicate +/−one standard deviation.

### Experiments A4, A5:

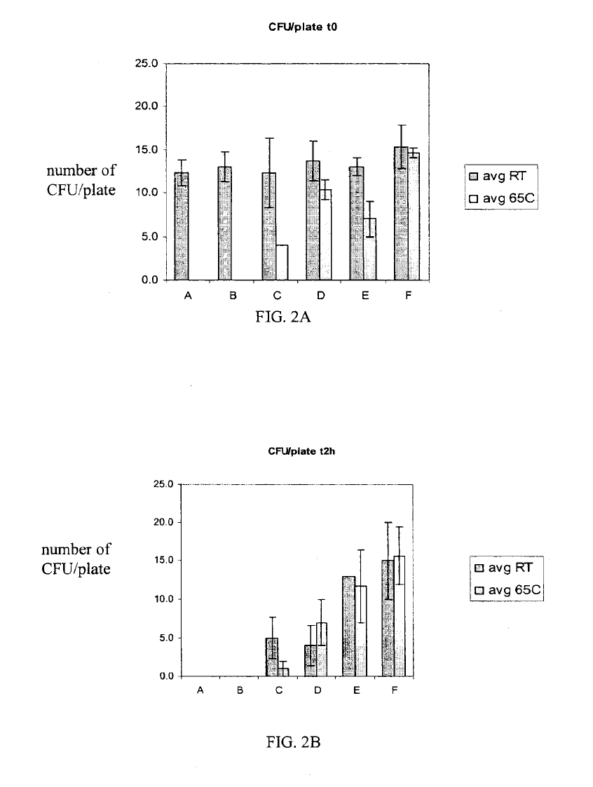

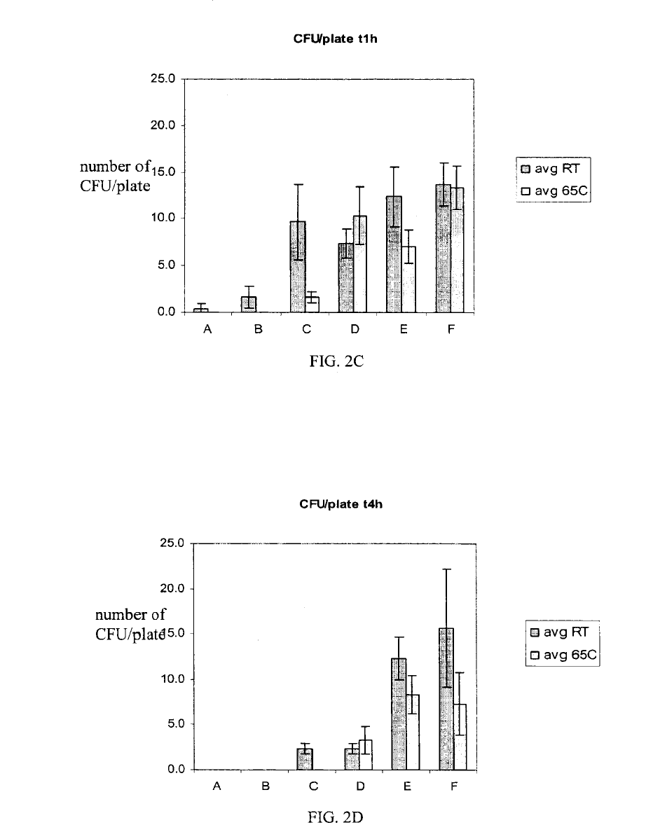

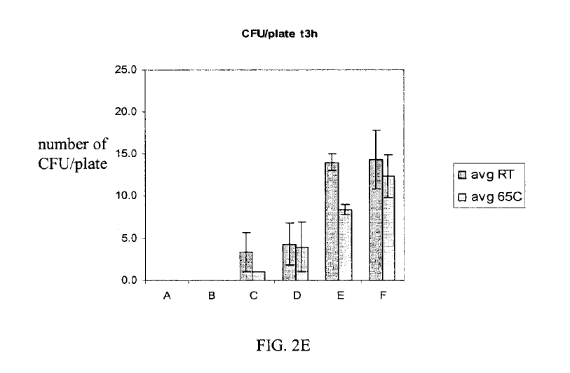

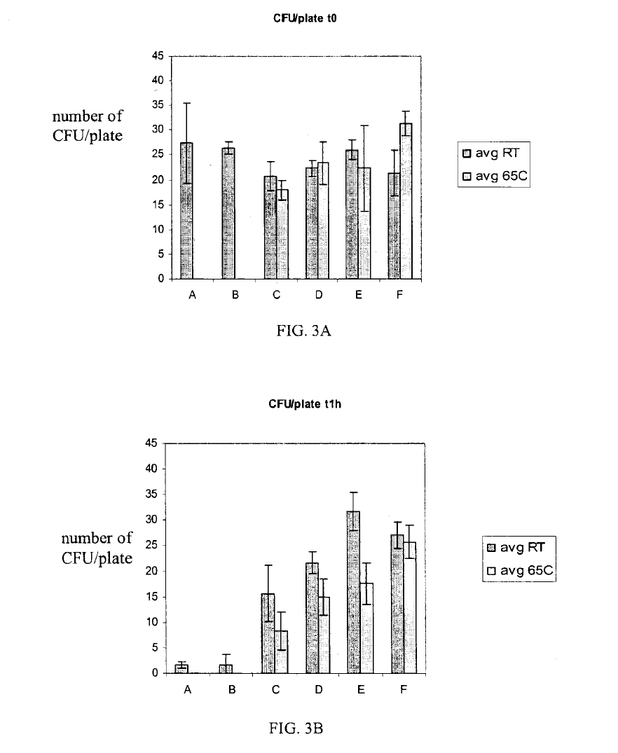

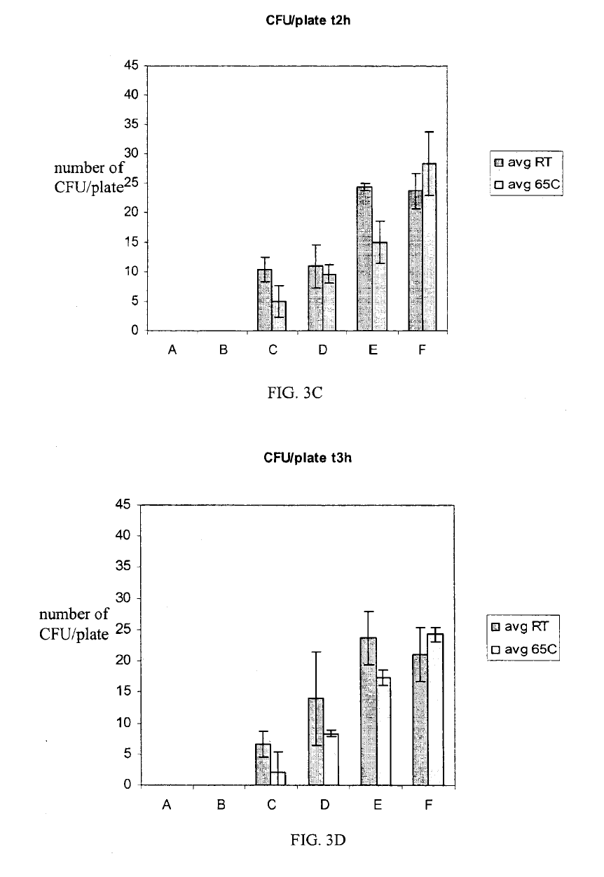

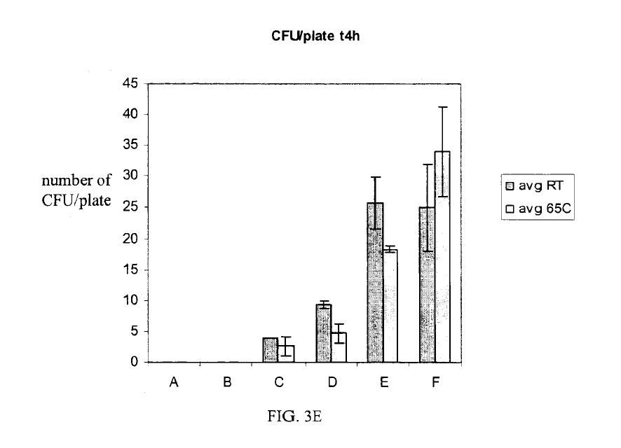

In experiment A4, 250 μl spore suspension (5×10⁴ spores) were added to 25 ml of the reagents. In experiment A5, 625 μl spore suspension (1.25×10⁵ spores) were added to 25 ml of the reagents. Sampling time points were t0, t1h, t2h, t3h, t4h, t5h. In order to differentiate whether CFU originated from vegetative cells or from spores, at each time point samples were split in two equivalent aliquots. One aliquot was subjected to heat treatment (65° C., 30 min) to kill vegetative cells before enumeration of residual heat-resistant spores. The other aliquot was plated at room temperature (RT). Average plate counts (FIGS. 2A-2E and 3A-3E, respectively) are based on counting three plates; error bars indicate +/−one standard deviation.

### Results:

### Experiment A3:

At t45 min recovery of CFUs from flasks A and B was reduced to 9% (1.7 CFU) and 43% (8 CFU), respectively, as compared to control flask F. At t90 min and t180 min zero colony forming units (CFU) were recovered from flasks A and B. For flasks C and D retrieval decreased over time but did not drop below 16% (reagent C) and 39% (reagent D) at 180 min. Recovery levels from the flask with reagent E did not decrease (Table 20).

TABLE 20
Experiment A3: CFU % recovery (as compared to control flask F)

| | 0 min | 10 min | 45 min | 90 min | 180 min |
|---|---|---|---|---|---|
| A | 85 | 81 | 9 | 0 | 0 |
| B | 121 | 66 | 43 | 0 | 0 |
| C | 142 | 77 | 82 | 48 | 16 |
| D | 108 | 81 | 55 | 64 | 39 |
| E | 119 | 65 | 94 | 144 | 95 |
| F | 100 | 100 | 100 | 100 | 100 |

### Experiments A4, A5:

In both experiments CFU recovery from flasks A and B at t0 and t1h originated from heat-sensitive cells because colony counts were zero for the samples which received heat treatment. No CFU were retrieved from flask A or B for t2h, t3h, t4h (FIGS. 2 and 3). For both reagents C and D % recovery decreased over time but of all compounds tested reagents A and B killed most effectively (Tables 2a, 2b, 3a, 3b). Reagent E was not more effective than the water control F (FIGS. 2 and 3).

TABLE 21
Experiment A4 absent heat: CFU % recovery
(as compared to control flask F) RT

| | 0 min | 1 h | 2 h | 3 h | 4 h |
|---|---|---|---|---|---|
| A | 81 | 2 | 0 | 0 | 0 |
| B | 85 | 12 | 0 | 0 | 0 |
| C | 81 | 71 | 33 | 23 | 15 |
| D | 89 | 54 | 27 | 30 | 15 |
| E | 85 | 90 | 87 | 98 | 79 |
| F | 100 | 100 | 100 | 100 | 100 |

TABLE 22
Experiment A4 with heat: CFU % recovery
(as compared to control flask F) 65° C.

| | 0 min | 1 h | 2 h | 3 h | 4 h |
|---|---|---|---|---|---|
| A | 0 | 0 | 0 | 0 | 0 |
| B | 0 | 0 | 0 | 0 | 0 |
| C | 27 | 13 | 6 | 8 | 0 |
| D | 70 | 78 | 45 | 33 | 46 |
| E | 48 | 53 | 74 | 68 | 114 |
| F | 100 | 100 | 100 | 100 | 100 |

TABLE 23
Experiment A5 absent heat: CFU % recovery
(as compared to control flask F) RT

| | 0 min | 1 h | 2 h | 3 h | 4 h |
|---|---|---|---|---|---|
| A | 128 | 6 | 0 | 0 | 0 |
| B | 124 | 6 | 0 | 0 | 0 |
| C | 97 | 58 | 44 | 32 | 16 |
| D | 105 | 80 | 46 | 67 | 37 |
| E | 122 | 117 | 103 | 113 | 103 |
| F | 100 | 100 | 100 | 100 | 100 |

TABLE 24
Experiment A5 with heat: CFU % recovery
(as compared to control flask F) 65° C.

| | 0 min | 1 h | 2 h | 3 h | 4 h |
|---|---|---|---|---|---|
| A | 0 | 0 | 0 | 0 | 0 |
| B | 0 | 0 | 0 | 0 | 0 |
| C | 58 | 32 | 18 | 8 | 8 |
| D | 75 | 58 | 34 | 34 | 14 |
| E | 71 | 69 | 53 | 71 | 54 |
| F | 100 | 100 | 100 | 100 | 100 |

### CONCLUSIONS

While reagents C and D in a 4-hour time frame had a negative effect on spore survival, neither one of these reagents was as effective in killing spores as reagents A and B. Reagent E was not different from the water control F.

Viable cell counts demonstrated that reagents A and B affected heat sensitivity of spores very quickly at the t0 time point suggesting induction of a break in spore dormancy. Chemical disinfectants which are not toxic and able to diminish resistance of spores to killing are potentially of great benefit.

1. An antimicrobial composition comprising:
a pharmaceutically acceptable surfactant, wherein the total concentration of surfactant in said composition is 0.05% to 3% by weight per volume in water; and
a monoprotic organic acid comprising a carbon backbone of 4 to 10 carbons, wherein the total concentration of acid in said antimicrobial composition is 0.3% to 3% by weight per volume in water.

2. The antimicrobial composition of claim 1 wherein said monoprotic organic acid has the general structure of: 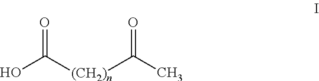
wherein n is an integer selected from 1 to 6.

3. The antimicrobial composition of claim 2, wherein said surfactant is an anionic surfactant.

4. The antimicrobial composition of claim 2, wherein said surfactant is selected from the group consisting of sodium dodecyl sulfate, sodium laureth sulfate, cetyl pyridinium chloride and benzalkonium chloride.

5. The antimicrobial composition of claim 1, wherein said surfactant is a cationic quaternary ammonium compound selected from the group consisting of benzalkonium chloride, cetylpyridinium bromide and cetylpyridinium chloride.

6. The antimicrobial composition of claim 1 formed as a foam having a cylinder foam test half life of at least ten minutes.

7. The antimicrobial composition of claim 2 wherein the surfactant is an ionic surfactant; and n is 2.

8. The antimicrobial composition of claim 7 wherein the surfactant is SDS.

9. The antimicrobial composition of claim 8 wherein the composition consists essentially of 0.05 to 1% by weight per volume in water SDS and 0.3 to 3% by weight per volume in water levulinic acid.

10. The antimicrobial composition of claim 8 wherein the composition consists essentially of 0.05 to 0.5% by weight per volume in water SDS and 0.3 to 2% by weight per volume in water levulinic acid.

11. A method of inactivating bacteria, said method comprising contacting the bacteria with the composition of claim 1.

12. A method of treating surfaces contaminated with bacteria, animal bodily fluids, waste or animal tissue, said method comprising contacting the surface with an aqueous composition comprising:
0.5% to 3% by weight per volume in water of an organic acid; and
0.05% to 2% by weight per volume in water of an ionic surfactant.

13. The method of claim 12 where in said organic acid is a monoprotic organic acid comprising a carbon backbone of 4 to 10 carbons.

14. The method of claim 12 where in said organic acid has the general structure of: 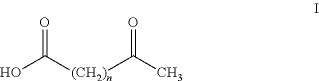
wherein n is an integer selected from 1 to 6, and said surfactant is selected from the group consisting of a quaternary ammonium cation, sodium dodecyl sulfate, sodium laureth sulfate, and cetyl pyridinium chloride.

15. The method of claim 14 wherein the acid is levulinic acid and the surfactant is sodium dodecyl sulfate or sodium laureth sulfate.

16. A method of treating produce, said method comprising the step of contacting the produce with a composition comprising an acid having the general structure of: 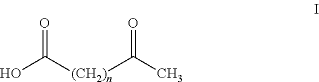
wherein n is an integer selected from 1 to 6, and
an anionic surfactant, wherein the total concentration of acid present in said composition is 0.3 to 1% by weight per volume in water and the total concentration of surfactant present in said composition is 0.05 to 0.5% by weight per volume in water.

17. The method of claim 16 wherein the acid is levulinic acid and the surfactant is SDS.

18. The method of claim 17 wherein the produce is sprayed with said composition.

19. The method of claim 17 wherein the produce is soaked in said composition for 1 to 5 minutes.

20. A method of treating eggs, said method comprising the step of contacting the eggs with an aqueous composition comprising:
0.5% to 3% by weight per volume in water of an organic acid; and
0.05% to 2% by weight per volume in water of an ionic surfactant.

21. The method of claim 20 wherein the eggs are sprayed with said composition.

22. The method of claim 20 wherein the eggs are soaked in said composition for 1 to 5 minutes.

23. Method for removing biofilms from a solid surface, said method comprising the steps of contacting the biofilm with an aqueous composition comprising:
0.5% to 3% by weight per volume in water of a monoprotic organic acid comprising a carbon backbone of 4 to 10 carbons; and
0.05% to 2% by weight per volume in water of an ionic surfactant.

24. The method of claim 23 where in said organic acid has the general structure of: 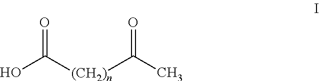
wherein n is an integer selected from 1 to 6, and said surfactant is selected from the group consisting of a quaternary ammonium cation, sodium dodecyl sulfate, sodium laureth sulfate, and cetyl pyridinium chloride.

25. The method of claim 24 wherein the acid is levulinic acid and the surfactant is sodium dodecyl sulfate or sodium laureth sulfate.

26. A method for decontaminating seeds, said method comprising the steps of contacting the seeds with an aqueous composition comprising:
0.5% to 3% by weight per volume in water of a monoprotic organic acid comprising a carbon backbone of 4 to 10 carbons; and
0.05% to 2% by weight per volume in water of an ionic surfactant.

27. The method of claim 26 where in said organic acid has the general structure of: 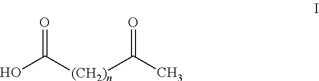
wherein n is an integer selected from 1 to 6, and said surfactant is selected from the group consisting of a quaternary ammonium cation, sodium dodecyl sulfate, sodium laureth sulfate, and cetyl pyridinium chloride.

28. The method of claim 27 wherein the acid is levulinic acid and the surfactant is sodium dodecyl sulfate or sodium laureth sulfate.

29. A method of providing a processed food with antibacterial qualities, said method comprising:
combining the composition of claim 7 with raw food material comprising to form a mixture; and processing said mixture to form said processed food.

30. The method of claim 29 wherein the composition of claim 7 is combined with unprocessed meats and said mixture is then ground.

31. The method of claim 29 wherein the composition of claim 7 is combined with shelved nuts to form a mixture, and the mixture is then ground for the preparation of nut butters.

32. The method of claim 29, wherein the composition of claim 7 is used as an additive to solutions packaged with a food.

33. A composition comprising:
a pharmaceutically acceptable surfactant; and
a monoprotic organic acid comprising a carbon backbone of 4 to 10 carbons,
wherein the pharmaceutically acceptable surfactant and the monoprotic organic acid are in a weight ratio of between about 1:60 to about 10:1.

34. The composition of claim 33, wherein said monoprotic organic acid has the general structure of: 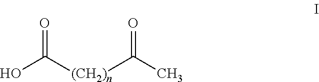
wherein n is an integer selected from 1 to 6.

35. The composition of claim 33, wherein said surfactant is selected from the group consisting of sodium dodecyl sulfate, sodium laureth sulfate, cetylpyridinium chloride, benzalkonium chloride, and cetylpyridinium bromide.

36. The composition of claim 33 wherein the composition consists essentially of a pharmaceutically acceptable surfactant; and a monoprotic organic acid, and wherein the pharmaceutically acceptable surfactant is SDS, and the monoprotic organic acid is levulinic acid.

37. The composition of claim 36, wherein the weight ratio of the SDS to the levulinic acid is between about 1:40 to about 5:3.
